(19) United States
(12) Patent Application Publication
(10) Pub. No.: US 2022/0195405 A1
(43) Pub. Date: Jun. 23, 2022

(54) ENGINEERED SSDNASE-FREE CRISPR ENDONUCLEASES

(71) Applicant: MONSANTO TECHNOLOGY LLC, St. Louis, MO (US)

(72) Inventors: Graeme S. GARVEY, Woodland, CA (US); Elysia K. KRIEGER, Kirkwood, MO (US)

(73) Assignee: MONSANTO TECHNOLOGY LLC, St. Louis, MO (US)

(21) Appl. No.: 17/552,431

(22) Filed: Dec. 16, 2021

Related U.S. Application Data
(60) Provisional application No. 63/126,983, filed on Dec. 17, 2020.

(51) Int. Cl. C12N 9/22; C12N 15/11; C12N 15/70; C12N 15/90

(52) U.S. Cl. CPC C12N 9/22; C12N 15/11; C12N 2310/20; C12N 15/902; C12N 15/70

(57) ABSTRACT

The present disclosure provides compositions related to engineered CRISPR endonuclease proteins that have a reduced ability to non-specifically cleave single-stranded DNA (ssDNA) as compared to its reference wildtype protein. This disclosure also provides methods related to the use of, and generation of, engineered CRISPR endonuclease proteins that have a reduced ability to non-specifically cleave ssDNA as compared to its reference wildtype protein.

### CROSS-REFERENCE TO RELATED APPLICATION AND INCORPORATION OF SEQUENCE LISTING

This application claims the benefit of U.S. Provisional Application No. 63/126,983, filed Dec. 17, 2020, which is incorporated by reference herein in its entirety. A sequence listing contained in the file named P34731US01_SL.txt, which is 185,550 bytes (measured in MS-Windows®) and created on Nov. 29, 2021, and comprises 23 sequences, is filed electronically herewith and incorporated by reference in its entirety.

### FIELD

The present disclosure relates to compositions and methods related to using engineered RNA-guided CRISPR endonucleases to reduce non-specific cleavage of single-stranded DNA (ssDNA). The present disclosure also relates to compositions and methods related to refining the concentration of magnesium to reduce non-specific cleavage of ssDNA by RNA-guided CRISPR endonucleases.

### BACKGROUND

CRISPR (clustered regularly interspaced short palindromic repeats) endonucleases (e.g., Cas9. CasX, Cas12a, CasY) are proteins guided by guide RNAs to a target nucleic acid molecule, where the endonuclease can then cleave one or two strands the target nucleic acid molecule. Recent reports have indicated that Cas12a (also referred to as Cpf1) can exhibit uncontrolled non-target cleavage of single-stranded DNA (ssDNA).

This disclosure demonstrates that CRISPR endonucleases can be modified to cleave double-stranded DNA (dsDNA) while reducing or eliminating their ability to non-specifically cleave ssDNA. This disclosure also demonstrates that the magnesium chloride concentration of a solution comprising a. CRISPR endonuclease can be manipulated to allow a CRISPR endonuclease to cleave dsDNA, while reducing or eliminating the CRISPR endonuclease's ability to non-specifically cleave ssDNA.

### BRIEF DESCRIPTION OF DRAWINGS

FIG. 1 depicts the expected sizes of template DNA cleaved by SpCas9 or LbCas12a.

FIG. 2 depicts a sequence alignment of a fragment of LbCas12a comprising the conserved R1138 residue along with homologues FnCas12a and AsCas12a. Conserved residues are shown in gray. Potential amino acid substitutions that can alter charge/change donor capacity are shown in italics, and amino acid residues affecting Mg²⁺ mediated ssDNAse activity are underlined. Positions of key amino acid residues are noted above the Wt sequence.

### SUMMARY

In one aspect, this disclosure provides an engineered RNA-guided CRISPR nuclease comprising at least one mutation in a DNA catalytic domain, where the engineered RNA guided CRISPR nuclease exhibits reduced non-specific cleavage of single-stranded DNA (ssDNA) as compared to the reference wildtype RNA-guided CRISPR nuclease lacking the at least one mutation.

In one aspect, this disclosure provides a method of creating an engineered RNA-guided CRISPR nuclease comprising editing a polynucleotide encoding a wildtype RNA-guided CRISPR nuclease to generate at least one mutation in a DNA catalytic domain, where the engineered RNA-guided CRISPR nuclease exhibits reduced non-specific cleavage of single-stranded DNA as compared to the wildtype RNA-guided CRISPR nuclease lacking the at least one mutation.

In one aspect, this disclosure provides a method of reducing non-specific single-stranded DNA (ssDNA) cleavage caused by an RNA-guided CRISPR nuclease, comprising providing a cell with an engineered RNA-guided CRISPR nuclease comprising at least one mutation in a DNA catalytic domain as compared to a reference wildtype RNA-guided CRISPR nuclease, where the engineered RNA guided CRISPR nuclease exhibits reduced non-specific cleavage of a non-target ssDNA as compared to the reference wildtype RNA-guided CRISPR nuclease lacking the at least one mutation.

In one aspect, this disclosure provides a method of reducing non-specific single-stranded DNA (ssDNA) cleavage caused by an RNA-guided CRISPR nuclease, comprising contacting an RNA-guided CRISPR nuclease with a non-target ssDNA in a test solution, where the test solution comprises MgCl₂ at a concentration of less than 10 mM, and wherein the non-specific ssDNA cleavage is reduced as compared to the non-specific ssDNA cleavage caused by the RNA-guided CRISPR nuclease in a control solution comprising MgCl₂ at a concentration of equal to or greater than 10 mM.

Several embodiments relate to an engineered RNA-guided CRISPR nuclease comprising at least one mutation in a DNA catalytic domain, wherein the engineered RNA guided CRISPR nuclease exhibits reduced non-specific cleavage of single-stranded DNA (ssDNA) as compared to the reference wildtype RNA-guided CRISPR nuclease lacking the at least one mutation. In some embodiments, the engineered RNA-guided CRISPR nuclease is part of a ribonucleoprotein. In some embodiments, the ribonucleoprotein comprises at least one guide nucleic acid. In some embodiments, the at least one guide nucleic acid comprises at least one guide RNA. In some embodiments, the at least one guide nucleic acid does not comprise a tracr. In some embodiments, the engineered RNA-guided CRISPR nuclease is a Cas12a nuclease. In some embodiments, the engineered RNA-guided CRISPR nuclease is selected from the group consisting of a Cas9 nuclease, a CasX nuclease, a CasY nuclease, and a C2c2 nuclease. In some embodiments, the engineered RNA-guided CRISPR nuclease comprises an amino acid sequence selected from the group consisting of SEQ ID NOs: 7-12. In some embodiments, the engineered RNA-guided CRISPR nuclease exhibits the ability to cleave double-stranded DNA (dsDNA). In some embodiments, the engineered RNA-guided CRISPR nuclease cleaves dsDNA at a rate that is at least 50% of the cleavage rate of the cleavage rate of the wildtype RNA-guided CRISPR nuclease. In some embodiments, the DNA catalytic domain comprises a RuvC domain, a Nuc domain, and/or an HNH domain. In some embodiments, at least one mutation in a DNA catalytic domain is selected from the group consisting of an insertion, a deletion, and a substitution. In some embodiments, at least one mutation in a DNA catalytic domain corresponds to a substitution of an amino acid at a position selected from the group consisting of position 925 and position 1138 as compared to SEQ ID NO: 2. In some embodiments, at least one mutation in a DNA catalytic domain corresponds to a substitution of an amino acid at a position corresponding to position 1138 as compared to SEQ ID NO: 2. In some embodiments, at least one mutation in a DNA catalytic domain corresponds to a substitution of an amino acid at a position corresponding to position 1146 as compared to SEQ ID NO: 2. In some embodiments, at least one mutation in a DNA catalytic domain corresponds to a substitution of an amino acid at a position corresponding to position 1148 as compared to SEQ ID NO: 2. In some embodiments, at least one mutation in a DNA catalytic domain corresponds to a substitution of an amino acid at a position corresponding to position 1218 of wtFnCas12a. In some embodiments, at least one mutation in a DNA catalytic domain corresponds to a substitution of an amino acid at a position corresponding to position 1225 of wtFnCas12a. In some embodiments, at least one mutation in a DNA catalytic domain corresponds to a substitution of an amino acid at a position corresponding to position 1227 of wtFnCas12a. In some embodiments, at least one mutation in a DNA catalytic domain corresponds to a substitution of an amino acid at a position corresponding to position 1226 of wtAsCas12a. In some embodiments, at least one mutation in a DNA catalytic domain corresponds to a substitution of an amino acid at a position corresponding to position 1234 of wtAsCas12a. In some embodiments, at least one mutation in a DNA catalytic domain corresponds to a substitution of an amino acid at a position corresponding to position 1235 of wtAsCas12a.

Several embodiments relate to a method of creating an engineered RNA-guided CRISPR nuclease comprising editing a polynucleotide encoding a wildtype RNA-guided CRISPR nuclease to generate at least one mutation in a DNA catalytic domain, wherein the engineered RNA-guided CRISPR nuclease exhibits reduced non-specific cleavage of single-stranded DNA as compared to the wildtype RNA-guided CRISPR nuclease lacking the at least one mutation. In some embodiments, the ribonucleoprotein comprises at least one guide nucleic acid. In some embodiments, the at least one guide nucleic acid comprises at least one guide RNA. In some embodiments, the at least one guide nucleic acid does not comprise a tracr. In some embodiments, the engineered RNA-guided CRISPR nuclease is a Cas12a nuclease. In some embodiments, the engineered RNA-guided CRISPR nuclease is selected from the group consisting of a Cas9 nuclease, a CasX nuclease, a CasY nuclease, and a C2c2 nuclease. In some embodiments, the engineered RNA-guided CRISPR nuclease comprises an amino acid sequence selected from the group consisting of SEQ ID NOs: 7-12. In some embodiments, the engineered RNA-guided CRISPR nuclease exhibits the ability to cleave double-stranded DNA (dsDNA). In some embodiments, the engineered RNA-guided CRISPR nuclease cleaves dsDNA at a rate that is at least 50% of the cleavage rate of the cleavage rate of the wildtype RNA-guided CRISPR nuclease. In some embodiments, the DNA catalytic domain comprises a RuvC domain, a Nuc domain, and/or an HNH domain. In some embodiments, at least one mutation in a DNA catalytic domain is selected from the group consisting of an insertion, a deletion, and a substitution. In some embodiments, at least one mutation in a DNA catalytic domain corresponds to a substitution of an amino acid at a position selected from the group consisting of position 925 and position 1138 as compared to SEQ ID NO: 2. In some embodiments, at least one mutation in a DNA catalytic domain corresponds to a substitution of an amino acid at a position corresponding to position 1138 as compared to SEQ ID NO: 2. In some embodiments, at least one mutation in a DNA catalytic domain corresponds to a substitution of an amino acid at a position corresponding to position 1146 as compared to SEQ ID NO: 2. In some embodiments, at least one mutation in a DNA catalytic domain corresponds to a substitution of an amino acid at a position corresponding to position 1148 as compared to SEQ ID NO: 2. In some embodiments, at least one mutation in a DNA catalytic domain corresponds to a substitution of an amino acid at a position corresponding to position 1218 of wtFnCas12a. In some embodiments, at least one mutation in a DNA catalytic domain corresponds to a substitution of an amino acid at a position corresponding to position 1225 of wtFnCas12a. In some embodiments, at least one mutation in a DNA catalytic domain corresponds to a substitution of an amino acid at a position corresponding to position 1227 of wtFnCas12a. In some embodiments, at least one mutation in a DNA catalytic domain corresponds to a substitution of an amino acid at a position corresponding to position 1226 of wtAsCas12a. In some embodiments, at least one mutation in a DNA catalytic domain corresponds to a substitution of an amino acid at a position corresponding to position 1234 of wtAsCas12a. In some embodiments, at least one mutation in a DNA catalytic domain corresponds to a substitution of an amino acid at a position corresponding to position 1235 of wtAsCas12a.

Several embodiments relate to a method of reducing non-specific single-stranded DNA (ssDNA) cleavage caused by an RNA-guided CRISPR nuclease, comprising providing a cell with an engineered RNA-guided CRISPR nuclease comprising at least one mutation in a DNA catalytic domain as compared to a reference wildtype RNA-guided CRISPR nuclease, wherein the engineered RNA guided CRISPR nuclease exhibits reduced non-specific cleavage of a non-target ssDNA as compared to the reference wildtype RNA-guided CRISPR nuclease lacking the at least one mutation. In some embodiments, the ribonucleoprotein comprises at least one guide nucleic acid. In some embodiments, the at least one guide nucleic acid comprises at least one guide RNA. In some embodiments, the at least one guide nucleic acid does not comprise a tracr. In some embodiments, the engineered RNA-guided CRISPR nuclease is a Cas12a nuclease. In some embodiments, the engineered RNA-guided CRISPR nuclease is selected from the group consisting of a Cas9 nuclease, a CasX nuclease, a CasY nuclease, and a C2c2 nuclease. In some embodiments, the engineered RNA-guided CRISPR nuclease comprises an amino acid sequence selected from the group consisting of SEQ ID NOs: 7-12. In some embodiments, the engineered RNA-guided CRISPR nuclease exhibits the ability to cleave double-stranded DNA (dsDNA). In some embodiments, the engineered RNA-guided CRISPR nuclease cleaves dsDNA at a rate that is at least 50% of the cleavage rate of the cleavage rate of the wildtype RNA-guided CRISPR nuclease. In some embodiments, the DNA catalytic domain comprises a RuvC domain, a Nuc domain, and/or an HNH domain. In some embodiments, at least one mutation in a DNA catalytic domain is selected from the group consisting of an insertion, a deletion, and a substitution. In some embodiments, at least one mutation in a DNA catalytic domain corresponds to a substitution of an amino acid at a position selected from the group consisting of position 925 and position 1138 as compared to SEQ ID NO: 2. In some embodiments, at least one mutation in a DNA catalytic domain corresponds to a substitution of an amino acid at a position corresponding to position 1138 as compared to SEQ ID NO: 2. In some embodiments, at least one mutation in a DNA catalytic domain corresponds to a substitution of an amino acid at a position corresponding to position 1146 as compared to SEQ ID NO: 2. In some embodiments, at least one mutation in a DNA catalytic domain corresponds to a substitution of an amino acid at a position corresponding to position 1148 as compared to SEQ ID NO: 2. In some embodiments, at least one mutation in a DNA catalytic domain corresponds to a substitution of an amino acid at a position corresponding to position 1218 of wtFnCas12a. In some embodiments, at least one mutation in a DNA catalytic domain corresponds to a substitution of an amino acid at a position corresponding to position 1225 of wtFnCas12a. In some embodiments, at least one mutation in a DNA catalytic domain corresponds to a substitution of an amino acid at a position corresponding to position 1227 of wtFnCas12a. In some embodiments, at least one mutation in a DNA catalytic domain corresponds to a substitution of an amino acid at a position corresponding to position 1226 of wtAsCas12a. In some embodiments, at least one mutation in a DNA catalytic domain corresponds to a substitution of an amino acid at a position corresponding to position 1234 of wtAsCas12a. In some embodiments, at least one mutation in a DNA catalytic domain corresponds to a substitution of an amino acid at a position corresponding to position 1235 of wtAsCas12a.

Several embodiments relate to a method of reducing non-specific single-stranded DNA (ssDNA) cleavage caused by an RNA-guided CRISPR nuclease, comprising contacting an RNA-guided CRISPR nuclease with a non-target ssDNA in a test solution, wherein the test solution comprises MgCl2 at a concentration of less than 10 mM, and wherein the non-specific ssDNA cleavage is reduced as compared to the non-specific ssDNA cleavage caused by the RNA-guided CRISPR nuclease in a control solution comprising MgCl2 at a concentration of equal to or greater than 10 mM. In some embodiments, the test solution comprises MgCl2 at a concentration of less than or equal to 5 mM. In some embodiments, the test solution comprises MgCl2 at a concentration of less than or equal to 0.02 mM. In some embodiments, the RNA-guided CRISPR nuclease is an engineered RNA-guided CRISPR nuclease comprising at least one mutation in a DNA catalytic domain as compared to the reference wildtype RNA-guided CRISPR nuclease lacking the at least one mutation. In some embodiments, the DNA catalytic domain is a RuvC domain or a Nuc domain. In some embodiments, the test solution is within a cell. In some embodiments, the cell is a prokaryotic cell or a eukaryotic cell. In some embodiments, the eukaryotic cell is a plant cell. In some embodiments, the RNA-guided CRISPR nuclease is a Cas12a nuclease. In some embodiments, the engineered RNA-guided CRISPR nuclease is an engineered Cas12a nuclease, and the wildtype RNA-guided CRISPR nuclease comprises the amino acid sequence of SEQ ID NO: 2. In some embodiments, the engineered RNA-guided CRISPR nuclease is selected from the group consisting of a Cas9 nuclease, a CasX nuclease, a CasY nuclease, and a C2c2 nuclease. In some embodiments, the engineered RNA-guided CRISPR nuclease comprises an amino acid sequence selected from the group consisting of SEQ ID NOs: 7-12.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York). The inventors do not intend to be limited to a mechanism or mode of action. Reference thereto is provided for illustrative purposes only.

The practice of this disclosure includes, unless otherwise indicated, conventional techniques of biochemistry, chemistry, molecular biology, microbiology, cell biology, plant biology, genomics, biotechnology, and genetics, which are within the skill of the art. See, for example, Green and Sambrook, Molecular Cloning: A Laboratory Manual, 4th edition (2012); Current Protocols In Molecular Biology (F. M. Ausubel, et al. eds., (1987)); Plant Breeding Methodology (N. F. Jensen, Wiley-Interscience (1988)); the series Methods In Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Animal Cell Culture (R. I. Freshney, ed. (1987)); Recombinant Protein Purification: Principles And Methods, 18-1142-75, GE Healthcare Life Sciences; C. N. Stewart, A. Touraev, V. Citovsky, T. Tzfira eds. (2011) Plant Transformation Technologies (Wiley-Blackwell); and R. H. Smith (2013) Plant Tissue Culture: Techniques and Experiments (Academic Press, Inc.).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc.

As used herein, terms in the singular and the singular forms “a,” “an,” and “the,” for example, include plural referents unless the content clearly dictates otherwise.

Any composition, nucleic acid molecule, polypeptide, cell, plant, etc. provided herein is specifically envisioned for use with any method provided herein.

In one aspect, this disclosure provides an engineered RNA-guided CRISPR nuclease comprising at least one mutation in a DNA catalytic domain, wherein the engineered RNA guided CRISPR nuclease exhibits reduced non-specific cleavage of single-stranded DNA (ssDNA) as compared to the reference wildtype RNA-guided CRISPR nuclease lacking the at least one mutation.

In another aspect, this disclosure provides a method of creating an engineered RNA-guided CRISPR nuclease comprising editing a polynucleotide encoding a wildtype RNA-guided CRISPR nuclease to generate at least one mutation in a DNA catalytic domain, wherein the engineered RNA-guided CRISPR nuclease exhibits reduced non-specific cleavage of ssDNA as compared to the wildtype RNA-guided CRISPR nuclease lacking the at least one mutation.

In a further aspect, this disclosure provides a method of reducing non-specific ssDNA cleavage caused by an RNA-guided CRISPR nuclease comprising providing a cell with an engineered RNA-guided CRISPR nuclease comprising at least one mutation in a DNA catalytic domain, wherein the engineered RNA guided CRISPR nuclease exhibits reduced non-specific cleavage of ssDNA as compared to the reference wildtype RNA-guided CRISPR nuclease lacking the at least one mutation.

As used herein, “cleavage” refers to the breakage of a phosphodiester bond between two nucleotides. If only one strand of a nucleic acid molecule is cleaved, such cleavage is referred to as “single-stranded cleavage.” If two strands of a nucleic acid molecule are cleaved, such cleavage is referred to as “double-stranded cleavage.” In an aspect, double-stranded cleavage produces blunt end cleavage products. Blunt-end cleavage products are produced when the two nucleic acid molecule strands are cleaved at the same position within the nucleic acid molecule. In another aspect, double-stranded cleavage produces overhanging cleavage products. Overhanging cleavage products are produced when the two nucleic acid molecule strands are cleaved at positions one or more nucleotides apart within the nucleic acid molecule.

### RNA-Guided CRISPR Nucleases

As used herein, an “RNA-guided CRISPR nuclease” refers to any nuclease derived from the CRISPR (clustered regularly interspaced short palindromic repeats) family of nucleases found in bacteria and archaea species. In an aspect, an RNA-guided CRISPR nuclease provided herein is an engineered RNA-guided CRISPR nuclease.

As used herein, an “engineered” RNA-guided CRISPR nuclease refers to an RNA-guided CRISPR nuclease comprising at least one non-naturally occurring mutation that is introduced to a wildtype RNA-guided CRISPR nuclease. A “wildtype RNA-guided CRISPR nuclease” refers to a naturally occurring, endogenous RNA-guided CRISPR nuclease found in an organism.

An engineered RNA-guided CRISPR nuclease can be created by modifying a wildtype RNA-guided CRISPR nuclease. Methods of editing polynucleotides that encode proteins are well known in the art. For example, a polynucleotide encoding a wildtype RNA-guided CRISPR nuclease can be modified by subjecting it to a mutagen (e.g., ethylmethane sulfonate (EMS), ionizing radiation) or a nuclease (e.g., a CRISPR nuclease, a zing-finger nuclease, a meganuclease, a transcription activator-like effector nuclease). A polynucleotide encoding a wildtype RNA-guided CRISPR nuclease can also be modified using other techniques standard in the art, such as, without being limiting, site-directed mutagenesis via PCR.

In an aspect, a polynucleotide encoding a wildtype RNA-guided CRISPR nuclease is edited to create an engineered RNA-guided CRISPR nuclease by subjecting the polynucleotide to a mutagen. As used herein, a “mutagen” refers to any agent that is capable of generating a modification, or mutation, to a nucleic acid sequence. In one aspect, a mutagen is a chemical mutagen. In one aspect, a mutagen is a physical mutagen. In another aspect, a mutagen is ionizing radiation. In another aspect, a mutagen is ultraviolet radiation. In another aspect, a mutagen is a reactive oxygen species. In another aspect, a mutagen is a deaminating agent. In another aspect, a mutagen is an alkylating agent. In another aspect, a mutagen is an aromatic amine. In another aspect, a mutagen is and intercalcating agent, such as ethidium bromide or proflavin. In another aspect, a mutagen is X-rays.

In an aspect, a chemical mutagen is selected from the group consisting of ethyl methanesulfonate (EMS), methyl methanesulfonate, diethylsulphonate, dimethyl sulfate, dimethyl sulfoxide, diethylnitrosamine, N-nitroso-N-methylurea, N-methyl-N-nitrosourea, N-nitroso-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, psoralen, benzene, Datura extract, bromodeoxyuridine, and beryllium oxide.

In another aspect, a polynucleotide encoding a wildtype RNA-guided CRISPR nuclease is edited to create an engineered RNA-guided CRISPR nuclease by subjecting the polynucleotide to a ribonucleoprotein. In another aspect, a polynucleotide encoding a wildtype RNA-guided CRISPR nuclease is edited to create an engineered RNA-guided CRISPR nuclease by subjecting the polynucleotide to a CRISPR nuclease. In another aspect, a polynucleotide encoding a wildtype RNA-guided CRISPR nuclease is edited to create an engineered RNA-guided CRISPR nuclease by subjecting the polynucleotide to a zinc-finger nuclease. In another aspect, a polynucleotide encoding a wildtype RNA-guided CRISPR nuclease is edited to create an engineered RNA-guided CRISPR nuclease by subjecting the polynucleotide to a TALEN. In another aspect, a polynucleotide encoding a wildtype RNA-guided CRISPR nuclease is edited to create an engineered RNA-guided CRISPR nuclease by subjecting the polynucleotide to a meganuclease.

In some embodiments, the RNA-guided CRISPR nuclease is a Class 1 RNA-guided CRISPR nuclease. In some embodiments, the RNA-guided CRISPR nuclease is a Class 1 RNA-guided CRISPR nuclease selected from the group consisting of Type I, Type IA, Type IB, Type IC, Type ID, Type IE, Type IF, Type IU, Type III, Type IIIA, Type IIIB, Type IIIC, Type MD, Type IV, Type IVA, Type IVB. In some embodiments, the RNA-guided CRISPR nuclease is a Class 2 CRISPR-Cas. In some embodiments, the RNA-guided CRISPR nuclease is a Class 2 RNA-guided CRISPR nuclease selected from the group consisting of Type II, Type IIA, Type IIB, Type IIC, Type V, Type VI.

In an aspect, an RNA-guided CRISPR nuclease is a Cas12a nuclease (also referred to as a Cpf1 nuclease). In another aspect, an RNA-guided CRISPR nuclease is a Cas9 nuclease. In another aspect, an RNA-guided CRISPR nuclease is a CasX nuclease. In another aspect, an RNA-guided CRISPR nuclease is a CasY nuclease. In another aspect, an RNA-guided CRISPR nuclease is a C2c2 nuclease. In an aspect, an RNA-guided CRISPR nuclease is selected from the group consisting of a Cas12a nuclease, a Cas9 nuclease, a CasX nuclease, a CasY nuclease, and a C2c2 nuclease.

In an aspect, an RNA-guided CRISPR nuclease is a Cas12a nuclease (also referred to as a Cpf1 nuclease). In an aspect, an RNA-guided CRISPR nuclease is a Lachnospiraceae bacterium Cas12a (LbCas12a) nuclease.

In another aspect, an engineered RNA-guided CRISPR nuclease is an engineered Cas9 nuclease. In another aspect, an engineered RNA-guided CRISPR nuclease is an engineered CasX nuclease. In another aspect, an engineered RNA-guided CRISPR nuclease is an engineered CasY nuclease. In another aspect, an engineered RNA-guided CRISPR nuclease is an engineered C2c2 nuclease. In an aspect, an engineered RNA-guided CRISPR nuclease is selected from the group consisting of an engineered Cas12a nuclease, an engineered Cas9 nuclease, an engineered CasX nuclease, an engineered CasY nuclease, and an engineered C2c2 nuclease.

In an aspect, an engineered RNA-guided CRISPR nuclease is an engineered Cas12a nuclease (also referred to as a Cpf1 nuclease). In an aspect, an engineered RNA-guided CRISPR nuclease is an engineered Lachnospiraceae bacterium Cas12a (LbCas12a) nuclease.

In an aspect, a Cas12a nuclease comprises an amino acid sequence at least 80% identical to SEQ ID NO: 2. In another aspect, a Cas12a nuclease comprises an amino acid sequence at least 85% identical to SEQ ID NO: 2. In another aspect, a Cas12a nuclease comprises an amino acid sequence at least 90% identical to SEQ ID NO: 2. In another aspect, a Cas12a nuclease comprises an amino acid sequence at least 95% identical to SEQ ID NO: 2. In another aspect, a Cas12a nuclease comprises an amino acid sequence 100% identical to SEQ ID NO: 2.

In an aspect, an engineered RNA-guided CRISPR nuclease comprises an amino acid sequence selected from the group consisting of SEQ ID Nos: 7, 8, 9, 10, 11, and 12.

In another aspect, an engineered Cas9 nuclease is derived from a bacteria genus selected from the group consisting of Streptococcus, Haloferax, Anabaena, Mycobacterium, Aeropyvrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacterium, Streptomyces, Aquifex, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myxococcus, Campylobacter, Wolinella, Acinetobacter, Envinia, Escherichia, Legionella, Methylococcus, Pasteurella, Photobacterium, Salmonella, Xanthomonas, Yersinia, Treponema, and Thermotoga.

In another aspect, an engineered Cas12a nuclease is derived from a bacteria genus selected from the group consisting of Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, Acidaminococcus, Peregrinibacteria, Butyrivibrio, Parcubacteria, Smithella, Candidatus, Moraxella, and Leptospira.

In an aspect, this disclosure provides a nucleic acid sequence encoding an engineered RNA-guided CRISPR nuclease provided herein.

When an RNA-guided CRISPR nuclease and a guide RNA form a complex, the whole system is called a “ribonucleoprotein.” The guide RNA guides the ribonucleoprotein to a complementary target sequence, where the CRISPR associated protein cleaves either one or two strands of DNA. Depending on the protein, cleavage can occur within a certain number of nucleotides (e.g., between 18-23 nucleotides for Cas12a) from a PAM site. PAM sites are only required for Type I and Type II CRISPR associated proteins; Type III CRISPR associated proteins do not require a PAM site for proper targeting or cleavage.

In an aspect, an engineered RNA-guided CRISPR nuclease provided herein is part of a ribonucleoprotein. In another aspect, a RNA-guided CRISPR nuclease provided herein is part of a ribonucleoprotein.

### Mutations

As used herein, a “mutation” refers to a non-naturally occurring alteration to a nucleic acid or amino acid sequence as compared to a naturally occurring reference nucleic acid or amino acid sequence from the same organism. It will be appreciated that, when identifying a mutation, the reference sequence should be from the same nucleic acid (e.g., gene, non-coding RNA) or amino acid (e.g., protein). As a non-limiting example, if an engineered LbCas12a nuclease comprising a mutation is compared to a wildtype sequence, then the wildtype sequence must be an endogenous LbCas12a sequence from the same species, not a homologous Cas12a sequence from a different bacteria species or a different RNA-guided CRISPR nuclease sequence (e.g., a Cas9 sequence). As used herein, a “wildtype” sequence refers to a naturally occurring amino acid or nucleotide sequence.

In an aspect, a mutation comprises the insertion of at least one nucleotide or amino acid. In another aspect, a mutation comprises the deletion of at least one nucleotide or amino acid. In a further aspect, a mutation comprises the substitution of at least one nucleotide or amino acid. In still a further aspect, a mutation comprises the inversion of at least two nucleotides or amino acids. In another aspect, a mutation is selected from the group consisting of an insertion, a deletion, and a substitution.

In an aspect, an engineered Cas12a nuclease comprises a substitution of the amino acid at position 925 as compared to SEQ ID NO: 2. In another aspect, an engineered Cas12a nuclease comprises a substitution of the amino acid at position 1138 as compared to SEQ ID NO: 2. In an aspect, an engineered Cas12a nuclease comprises a deletion of the amino acid at position 925 as compared to SEQ ID NO: 2. In another aspect, an engineered Cas12a nuclease comprises a deletion of the amino acid at position 1138 as compared to SEQ ID NO: 2. In an aspect, an engineered Cas12a nuclease comprises an insertion of at least one amino acid at amino acid position 925 as compared to SEQ ID NO: 2. In another aspect, an engineered Cas12a nuclease comprises an insertion of at least one amino acid at amino acid position 1138 as compared to SEQ ID NO: 2.

### DNA Catalytic Domain

As used herein, a “DNA catalytic domain” refers to a domain (or region) of an amino acid sequence that can affect cleavage of a nucleic acid molecule.

In an aspect, an engineered RNA-guided CRISPR nuclease comprises at least one mutation in a DNA catalytic domain. In another aspect, an engineered RNA-guided CRISPR nuclease comprises at least two mutations in a DNA catalytic domain. In another aspect, an engineered RNA-guided CRISPR nuclease comprises at least three mutations in a DNA catalytic domain. In another aspect, an engineered RNA-guided CRISPR nuclease comprises at least one mutation in each of at least two DNA catalytic domains.

In an aspect, a DNA catalytic domain comprises a RuvC domain.

In Cas9 and similar proteins, RuvC domains can comprise three discontinuous regions (RuvC-I, RuvC-II, and RuvC-III) with an HNH domain inserted between RuvC-II and RuvC-III. All three RuvC regions contribute to the nuclease activity of RuvC. In Cas9, RuvC cleaves the non-targeted strand of a double-stranded nucleic acid. RuvC domains comprise a six-stranded beta sheet surrounded by four alpha helices. RuvC domains are characterized by InterPro as belonging to the Pfam number PF18541. See, for example, RuvC endonuclease subdomain 3 at www(dot)ebi(dot)ac(dot)uk/interpro/entry/IPR041383.

Alternatively, in Cas12a, the RuvC domain comprises a Nuc domain and an arginine-rich bridge helix domain. See, for example, Cas12a, RuvC nuclease domain at www(dot)ebi(dot)ac(dot)uk/interpro/entry/IPR040852. In an aspect, a Nuc domain is positioned between a RuvC-II domain and a RuvC-III domain.

In an aspect, a DNA catalytic domain comprises a Nuc domain. See, for example, Cas12a nuclease domain at www(dot)ebi(dot)ac(dot)uk/interpro/entry/IPR040882.

In an aspect, a RuvC domain comprises a RuvC-I domain, a RuvC-II domain, a RuvC-III domain, or any combination thereof. In an aspect, a RuvC domain further comprises an HNH domain. In an aspect, a RuvC domain comprises an HNH domain between a RuvC-II domain and a RuvC-III domain. In another aspect, a RuvC domain further comprises a Nuc domain. In another aspect, a RuvC domain further comprises an arginine-rich bridge helix domain.

In an aspect, a DNA catalytic domain comprises an HNH domain. In Cas9, HNH cleaves the targeted strand of a double-stranded nucleic acid. See, for example HNH nuclease at www(dot)ebi(dot)ac(dot)uk/interpro/entry/IPR003615.

In an aspect, an engineered RNA-guided CRISPR nuclease comprises at least one mutation in a RuvC domain. In another aspect, an engineered RNA-guided CRISPR nuclease comprises at least one mutation in a RuvC-I domain. In another aspect, an engineered RNA-guided CRISPR nuclease comprises at least one mutation in a RuvC-II domain. In another aspect, an engineered RNA-guided CRISPR nuclease comprises at least one mutation in a RuvC-III domain. In another aspect, an engineered RNA-guided CRISPR nuclease comprises at least one mutation in a Nuc domain. In another aspect, an engineered RNA-guided CRISPR nuclease comprises at least one mutation in an HNH domain. In a further aspect, an engineered RNA-guided CRISPR nuclease comprises any combination of mutations in a RuvC-I domain, a RuvC-II domain, a RuvC-III domain, a Nuc domain, or an HNH domain.

Reduced Non-Specific Cleavage of ssDNA

In an aspect, an engineered RNA-guided CRISPR nuclease exhibits the ability to cleave target double-stranded DNA (dsDNA).

In an aspect, an engineered RNA-guided CRISPR nucleases cleaves target dsDNA at the same rate as its reference wildtype RNA-guided CRISPR nuclease. In another aspect, an engineered RNA-guided CRISPR nuclease cleaves target dsDNA at a rate that is at least 95% of the dsDNA cleavage rate of its reference wildtype RNA-guided CRISPR nuclease. In another aspect, an engineered RNA-guided CRISPR nuclease cleaves target dsDNA at a rate that is at least 90% of the dsDNA cleavage rate of its reference wildtype RNA-guided CRISPR nuclease. In another aspect, an engineered RNA-guided CRISPR nuclease cleaves target dsDNA at a rate that is at least 80% of the dsDNA cleavage rate of its reference wildtype RNA-guided CRISPR nuclease. In another aspect, an engineered RNA-guided CRISPR nuclease cleaves target dsDNA at a rate that is at least 70% of the dsDNA cleavage rate of its reference wildtype RNA-guided CRISPR nuclease. In another aspect, an engineered RNA-guided CRISPR nuclease cleaves target dsDNA at a rate that is at least 60% of the dsDNA cleavage rate of its reference wildtype RNA-guided CRISPR nuclease. In another aspect, an engineered RNA-guided CRISPR nuclease cleaves target dsDNA at a rate that is at least 50% of the dsDNA cleavage rate of its reference wildtype RNA-guided CRISPR nuclease. In another aspect, an engineered RNA-guided CRISPR nuclease cleaves target dsDNA at a rate that is at least 40% of the dsDNA cleavage rate of its reference wildtype RNA-guided CRISPR nuclease. In another aspect, an engineered RNA-guided CRISPR nuclease cleaves target dsDNA at a rate that is at least 30% of the dsDNA cleavage rate of its reference wildtype RNA-guided CRISPR nuclease. In another aspect, an engineered RNA-guided CRISPR nuclease cleaves target dsDNA at a rate that is at least 25% of the dsDNA cleavage rate of its reference wildtype RNA-guided CRISPR nuclease. In another aspect, an engineered RNA-guided CRISPR nuclease cleaves target dsDNA at a rate that is at least 20% of the dsDNA cleavage rate of its reference wildtype RNA-guided CRISPR nuclease. In another aspect, an engineered RNA-guided CRISPR nuclease cleaves target dsDNA at a rate that is at least 15% of the dsDNA cleavage rate of its reference wildtype RNA-guided CRISPR nuclease. In another aspect, an engineered RNA-guided CRISPR nuclease cleaves target dsDNA at a rate that is at least 10% of the dsDNA cleavage rate of its reference wildtype RNA-guided CRISPR nuclease. In another aspect, an engineered RNA-guided CRISPR nuclease cleaves target dsDNA at a rate that is at least 5% of the dsDNA cleavage rate of its reference wildtype RNA-guided CRISPR nuclease. In another aspect, an engineered RNA-guided CRISPR nuclease cleaves target dsDNA at a rate that is at least 1% of the dsDNA cleavage rate of its reference wildtype RNA-guided CRISPR nuclease.

In an aspect, an engineered RNA-guided CRISPR nuclease cleaves target dsDNA at a rate that is between 1% and 95% of the dsDNA cleavage rate of its reference wildtype RNA-guided CRISPR nuclease. In another aspect, an engineered RNA-guided CRISPR nuclease cleaves target dsDNA at a rate that is between 5% and 95% of the dsDNA cleavage rate of its reference wildtype RNA-guided CRISPR nuclease. In another aspect, an engineered RNA-guided CRISPR nuclease cleaves target dsDNA at a rate that is between 10% and 95% of the dsDNA cleavage rate of its reference wildtype RNA-guided CRISPR nuclease. In another aspect, an engineered RNA-guided CRISPR nuclease cleaves target dsDNA at a rate that is between 25% and 95% of the dsDNA cleavage rate of its reference wildtype RNA-guided CRISPR nuclease. In another aspect, an engineered RNA-guided CRISPR nuclease cleaves target dsDNA at a rate that is between 50% and 95% of the dsDNA cleavage rate of its reference wildtype RNA-guided CRISPR nuclease. In another aspect, an engineered RNA-guided CRISPR nuclease cleaves target dsDNA at a rate that is between 75% and 95% of the dsDNA cleavage rate of its reference wildtype RNA-guided CRISPR nuclease. In another aspect, an engineered RNA-guided CRISPR nuclease cleaves target dsDNA at a rate that is between 1% and 50% of the dsDNA cleavage rate of its reference wildtype RNA-guided CRISPR nuclease. In another aspect, an engineered RNA-guided CRISPR nuclease cleaves target dsDNA at a rate that is between 1% and 35% of the dsDNA cleavage rate of its reference wildtype RNA-guided CRISPR nuclease. In another aspect, an engineered RNA-guided CRISPR nuclease cleaves target dsDNA at a rate that is between 1% and 25% of the dsDNA cleavage rate of its reference wildtype RNA-guided CRISPR nuclease. In another aspect, an engineered RNA-guided CRISPR nuclease cleaves target dsDNA at a rate that is between 1% and 15% of the dsDNA cleavage rate of its reference wildtype RNA-guided CRISPR nuclease. In another aspect, an engineered RNA-guided CRISPR nuclease cleaves target dsDNA at a rate that is between 1% and 10% of the dsDNA cleavage rate of its reference wildtype RNA-guided CRISPR nuclease. In another aspect, an engineered RNA-guided CRISPR nuclease cleaves target dsDNA at a rate that is between 5% and 35% of the dsDNA cleavage rate of its reference wildtype RNA-guided CRISPR nuclease. In another aspect, an engineered RNA-guided CRISPR nuclease cleaves target dsDNA at a rate that is between 5% and 15% of the dsDNA cleavage rate of its reference wildtype RNA-guided CRISPR nuclease.

In an aspect, a wildtype RNA-guided CRISPR nuclease non-specifically cleaves a non-target ssDNA. As used herein, “non-specific cleavage” or “non-specifically cleave” refers to when an RNA-guided CRISPR nuclease cleaves a nucleic acid sequence that is not complementary to the nuclease's guide RNA. As used herein, “non-target ssDNA” refers to a ssDNA molecule that is not complementary to a guide nucleic acid.

In an aspect, an engineered RNA-guided CRISPR nuclease cannot non-specifically cleave a non-target ssDNA. In an aspect, an engineered RNA-guided CRISPR nuclease comprises a reduced ability to non-specifically cleave a non-target ssDNA as compared to its reference wildtype RNA-guided CRISPR nuclease. In an aspect, an engineered RNA-guided CRISPR nuclease comprises a DNA catalytic domain that cannot non-specifically cleave a non-target ssDNA. In another aspect, an engineered RNA-guided CRISPR nuclease comprises a DNA catalytic domain that comprises reduced ability to non-specifically cleave a non-target ssDNA as compared to its reference wildtype RNA-guided CRISPR nuclease.

In an aspect, an engineered RNA-guided CRISPR nuclease exhibits no detectable non-specific cleavage of ssDNA. ssDNA cleavage can be detected, for example, by isolating ssDNA that was exposed to the engineered RNA-guided CRISPR nuclease for at least 180 minutes at 37° C. and running the isolated DNA on an agarose gel to detect cleavage fragments. If no cleavage fragments are observed, one of ordinary skill in the art would determine that the engineered RNA-guided CRISPR nuclease exhibits no detectable cleavage of ssDNA.

In another aspect, an engineered RNA-guided CRISPR nuclease exhibits a reduced rate of non-specific ssDNA cleavage as compared to its reference wildtype RNA-guided CRISPR nuclease.

In an aspect, an engineered RNA-guided CRISPR nuclease non-specifically cleaves a non-target ssDNA at a rate that is less than 95% of the non-specific cleavage rate of its reference wildtype RNA-guided CRISPR nuclease on the same non-target ssDNA. In another aspect, an engineered RNA-guided CRISPR nuclease non-specifically cleaves a non-target ssDNA at a rate that is less than 90% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is less than 80% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is less than 70% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is less than 60% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is less than 50% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is less than 40% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is less than 30% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is less than 25% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is less than 20% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is less than 15% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is less than 10% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is less than 5% of the non-specific cleavage rate of its reference wildtype RNA-guided CRISPR nuclease on the same non-target ssDNA

In an aspect, an engineered RNA-guided CRISPR nuclease non-specifically cleaves a non-target ssDNA at a rate that is between 1% and 95% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is between 5% and 95% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is between 10% and 95% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is between 25% and 95% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is between 50% and 95% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is between 75% and 95% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is between 1% and 50% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is between 1% and 35% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is between 1% and 25% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is between 1% and 15% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is between 1% and 10% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is between 5% and 35% of the non-specific cleavage rate of its reference wildtype RNA-guided CRISPR nuclease on the same non-target ssDNA In another aspect, an engineered RNA-guided CRISPR nuclease non-specifically cleaves a non-target ssDNA at a rate that is between 5% and 15% of the non-specific cleavage rate of its reference wildtype RNA-guided CRISPR nuclease on the same non-target ssDNA

The rate of ssDNA or dsDNA cleavage can be measured by providing a known amount of ssDNA or dsDNA to an engineered RNA-guided CRISPR nuclease or its reference wildtype RNA-guided CRISPR nuclease for a specific amount of time, and then determining how much of the original ssDNA or dsDNA remained intact and how much of the original ssDNA dsDNA was cleaved.

In an aspect, a rate of ssDNA or dsDNA cleavage is measured within 180 minutes of introducing an engineered RNA-guided CRISPR nuclease to a ssDNA or dsDNA molecule. In another aspect, a rate of ssDNA or dsDNA cleavage is measured within 150 minutes of introducing an engineered RNA-guided CRISPR nuclease to a ssDNA or dsDNA molecule. In another aspect, a rate of ssDNA or dsDNA cleavage is measured within 120 minutes of introducing an engineered RNA-guided CRISPR nuclease to a ssDNA or dsDNA molecule. In another aspect, a rate of ssDNA or dsDNA cleavage is measured within 90 minutes of introducing an engineered RNA-guided CRISPR nuclease to a ssDNA or dsDNA molecule. In another aspect, a rate of ssDNA or dsDNA cleavage is measured within 60 minutes of introducing an engineered RNA-guided CRISPR nuclease to a ssDNA or dsDNA molecule. In another aspect, a rate of ssDNA or dsDNA cleavage is measured within 30 minutes of introducing an engineered RNA-guided CRISPR nuclease to a ssDNA or dsDNA molecule. In another aspect, a rate of ssDNA or dsDNA cleavage is measured within 15 minutes of introducing an engineered RNA-guided CRISPR nuclease to a ssDNA or dsDNA molecule. In another aspect, a rate of ssDNA or dsDNA cleavage is measured within 10 minutes of introducing an engineered RNA-guided CRISPR nuclease to a ssDNA or dsDNA molecule.

In an aspect, a rate of ssDNA or dsRNA cleavage is measured where the cleavage occurs at a temperature of less than 45° C. In another aspect, a rate of ssDNA or dsRNA cleavage is measured where the cleavage occurs at a temperature of less than 42° C. In another aspect, a rate of ssDNA or dsRNA cleavage is measured where the cleavage occurs at a temperature of less than 40° C. In another aspect, a rate of ssDNA or dsRNA cleavage is measured where the cleavage occurs at a temperature of less than 37° C. In another aspect, a rate of ssDNA or dsRNA cleavage is measured where the cleavage occurs at a temperature of less than 35° C. In another aspect, a rate of ssDNA or dsRNA cleavage is measured where the cleavage occurs at a temperature of less than 30° C. In another aspect, a rate of ssDNA or dsRNA cleavage is measured where the cleavage occurs at a temperature of less than 25° C.

In another aspect, a rate of ssDNA or dsRNA cleavage is measured where the cleavage occurs at a temperature of at least 20° C. In another aspect, a rate of ssDNA or dsRNA cleavage is measured where the cleavage occurs at a temperature of at least 25° C. In another aspect, a rate of ssDNA or dsRNA cleavage is measured where the cleavage occurs at a temperature of at least 30° C. In another aspect, a rate of ssDNA or dsRNA cleavage is measured where the cleavage occurs at a temperature of at least 35° C. In another aspect, a rate of ssDNA or dsRNA cleavage is measured where the cleavage occurs at a temperature of at least 37° C. In another aspect, a rate of ssDNA or dsRNA cleavage is measured where the cleavage occurs at a temperature of at least 40° C. In another aspect, a rate of ssDNA or dsRNA cleavage is measured where the cleavage occurs at a temperature of at least 42° C.

In another aspect, a rate of ssDNA or dsRNA cleavage is measured where the cleavage occurs at a temperature of between 20° C. and 45° C. In another aspect, a rate of ssDNA or dsRNA cleavage is measured where the cleavage occurs at a temperature of between 20° C. and 40° C. In another aspect, a rate of ssDNA or dsRNA cleavage is measured where the cleavage occurs at a temperature of between 20° C. and 37° C. In another aspect, a rate of ssDNA or dsRNA cleavage is measured where the cleavage occurs at a temperature of between 20° C. and 35° C. In another aspect, a rate of ssDNA or dsRNA cleavage is measured where the cleavage occurs at a temperature of between 20° C. and 30° C. In another aspect, a rate of ssDNA or dsRNA cleavage is measured where the cleavage occurs at a temperature of between 25° C. and 45° C. In another aspect, a rate of ssDNA or dsRNA cleavage is measured where the cleavage occurs at a temperature of between 25° C. and 40° C. In another aspect, a rate of ssDNA or dsRNA cleavage is measured where the cleavage occurs at a temperature of between 25° C. and 37° C. In another aspect, a rate of ssDNA or dsRNA cleavage is measured where the cleavage occurs at a temperature of between 25° C. and 35° C. In another aspect, a rate of ssDNA or dsRNA cleavage is measured where the cleavage occurs at a temperature of between 30° C. and 45° C. In another aspect, a rate of ssDNA or dsRNA cleavage is measured where the cleavage occurs at a temperature of between 30° C. and 40° C. In another aspect, a rate of ssDNA or dsRNA cleavage is measured where the cleavage occurs at a temperature of between 30° C. and 37° C. In another aspect, a rate of ssDNA or dsRNA cleavage is measured where the cleavage occurs at a temperature of between 35° C. and 42° C.

In an aspect, a rate of ssDNA or dsDNA cleavage is measured between 5 minutes and 300 minutes of introducing an engineered RNA-guided CRISPR nuclease to a ssDNA or dsDNA molecule. In another aspect, a rate of ssDNA or dsDNA cleavage is measured between 5 minutes and 250 minutes of introducing an engineered RNA-guided CRISPR nuclease to a ssDNA or dsDNA molecule. In another aspect, a rate of ssDNA or dsDNA cleavage is measured between 5 minutes and 200 minutes of introducing an engineered RNA-guided CRISPR nuclease to a ssDNA or dsDNA molecule. In another aspect, a rate of ssDNA or dsDNA cleavage is measured between 5 minutes and 180 minutes of introducing an engineered RNA-guided CRISPR nuclease to a ssDNA or dsDNA molecule. In another aspect, a rate of ssDNA or dsDNA cleavage is measured between 5 minutes and 150 minutes of introducing an engineered RNA-guided CRISPR nuclease to a ssDNA or dsDNA molecule. In another aspect, a rate of ssDNA or dsDNA cleavage is measured between 5 minutes and 120 minutes of introducing an engineered RNA-guided CRISPR nuclease to a ssDNA or dsDNA molecule. In another aspect, a rate of ssDNA or dsDNA cleavage is measured between 5 minutes and 90 minutes of introducing an engineered RNA-guided CRISPR nuclease to a ssDNA or dsDNA molecule. In another aspect, a rate of ssDNA or dsDNA cleavage is measured between 5 minutes and 60 minutes of introducing an engineered RNA-guided CRISPR nuclease to a ssDNA or dsDNA molecule. In another aspect, a rate of ssDNA or dsDNA cleavage is measured between 5 minutes and 30 minutes of introducing an engineered RNA-guided CRISPR nuclease to a ssDNA or dsDNA molecule. In another aspect, a rate of ssDNA or dsDNA cleavage is measured between 5 minutes and 15 minutes of introducing an engineered RNA-guided CRISPR nuclease to a ssDNA or dsDNA molecule.

In an aspect, a rate of ssDNA or dsDNA cleavage is measured within 180 minutes of introducing an RNA-guided CRISPR nuclease to a ssDNA or dsDNA molecule. In another aspect, a rate of ssDNA or dsDNA cleavage is measured within 150 minutes of introducing an RNA-guided CRISPR nuclease to a ssDNA or dsDNA molecule. In another aspect, a rate of ssDNA or dsDNA cleavage is measured within 120 minutes of introducing an RNA-guided CRISPR nuclease to a ssDNA or dsDNA molecule. In another aspect, a rate of ssDNA or dsDNA cleavage is measured within 90 minutes of introducing an RNA-guided CRISPR nuclease to a ssDNA or dsDNA molecule. In another aspect, a rate of ssDNA or dsDNA cleavage is measured within 60 minutes of introducing an RNA-guided CRISPR nuclease to a ssDNA or dsDNA molecule. In another aspect, a rate of ssDNA or dsDNA cleavage is measured within 30 minutes of introducing an RNA-guided CRISPR nuclease to a ssDNA or dsDNA molecule. In another aspect, a rate of ssDNA or dsDNA cleavage is measured within 15 minutes of introducing an RNA-guided CRISPR nuclease to a ssDNA or dsDNA molecule. In another aspect, a rate of ssDNA or dsDNA cleavage is measured within 10 minutes of introducing an RNA-guided CRISPR nuclease to a ssDNA or dsDNA molecule.

In an aspect, a rate of ssDNA or dsDNA cleavage is measured between 5 minutes and 300 minutes of introducing an RNA-guided CRISPR nuclease to a ssDNA or dsDNA molecule. In another aspect, a rate of ssDNA or dsDNA cleavage is measured between 5 minutes and 250 minutes of introducing an RNA-guided CRISPR nuclease to a ssDNA or dsDNA molecule. In another aspect, a rate of ssDNA or dsDNA cleavage is measured between 5 minutes and 200 minutes of introducing an RNA-guided CRISPR nuclease to a ssDNA or dsDNA molecule. In another aspect, a rate of ssDNA or dsDNA cleavage is measured between 5 minutes and 180 minutes of introducing an RNA-guided CRISPR nuclease to a ssDNA or dsDNA molecule. In another aspect, a rate of ssDNA or dsDNA cleavage is measured between 5 minutes and 150 minutes of introducing an RNA-guided CRISPR nuclease to a ssDNA or dsDNA molecule. In another aspect, a rate of ssDNA or dsDNA cleavage is measured between 5 minutes and 120 minutes of introducing an RNA-guided CRISPR nuclease to a ssDNA or dsDNA molecule. In another aspect, a rate of ssDNA or dsDNA cleavage is measured between 5 minutes and 90 minutes of introducing an RNA-guided CRISPR nuclease to a ssDNA or dsDNA molecule. In another aspect, a rate of ssDNA or dsDNA cleavage is measured between 5 minutes and 60 minutes of introducing an RNA-guided CRISPR nuclease to a ssDNA or dsDNA molecule. In another aspect, a rate of ssDNA or dsDNA cleavage is measured between 5 minutes and 30 minutes of introducing an RNA-guided CRISPR nuclease to a ssDNA or dsDNA molecule. In another aspect, a rate of ssDNA or dsDNA cleavage is measured between 5 minutes and 15 minutes of introducing an RNA-guided CRISPR nuclease to a ssDNA or dsDNA molecule.

In an aspect, an engineered RNA-guided CRISPR nuclease cleaves a dsDNA molecule in vivo. In another aspect, an engineered RNA-guided CRISPR nuclease cleaves a ssDNA molecule in vivo. In an aspect, an engineered RNA-guided CRISPR nuclease cleaves a dsDNA molecule in vitro. In another aspect, an engineered RNA-guided CRISPR nuclease cleaves a ssDNA molecule in vitro. In an aspect, an engineered RNA-guided CRISPR nuclease cleaves a dsDNA molecule ex vivo. In another aspect, an engineered RNA-guided CRISPR nuclease cleaves a ssDNA molecule ex vivo.

In an aspect, an RNA-guided CRISPR nuclease cleaves a dsDNA molecule in vivo. In another aspect, an RNA-guided CRISPR nuclease cleaves a ssDNA molecule in vivo. In an aspect, an RNA-guided CRISPR nuclease cleaves a dsDNA molecule in vitro. In another aspect, an RNA-guided CRISPR nuclease cleaves a ssDNA molecule in vitro. In an aspect, an RNA-guided CRISPR nuclease cleaves a dsDNA molecule ex vivo. In another aspect, an RNA-guided CRISPR nuclease cleaves a ssDNA molecule ex vivo.

As used herein, “in vivo” refers to within a living cell, tissue, or organism. As used herein, “in vitro” refers to within a labware. Non-limiting examples of labware include a test tube, a flask, a beaker, a graduated cylinder, a pipette, a petri dish, and a microtiter plate. As used herein, “ex vivo” refers to in a cell or tissue from an organism in an external environment. As a non-limiting example, a plant protoplast in a petri dish or test tube would be considered ex vivo.

### Magnesium

DNA catalytic domains often require magnesium for proper function. In an aspect, this disclosure provides a method of reducing ssDNA cleavage caused by an RNA-guided CRISPR nuclease comprising contacting a RNA-guided CRISPR nuclease with a target site in a solution, wherein the solution comprises MgCl₂ at a concentration of less than 10 mM, and wherein the reduced ssDNA cleavage is as compared to cleavage caused by the RNA-guided CRISPR nuclease in a control solution comprising MgCl₂ at a concentration of equal to or greater than 10 mM.

In another aspect, this disclosure provides a method of reducing ssDNA cleavage caused by an RNA-guided CRISPR nuclease comprising contacting a RNA-guided CRISPR nuclease with a target site in a solution, wherein the solution comprises Mg′ at a concentration of less than 10 mM, and wherein the reduced ssDNA cleavage is as compared to cleavage caused by the RNA-guided CRISPR nuclease in a control solution comprising Mg′ at a concentration of equal to or greater than 10 mM.

In an aspect, a solution comprises MgCl₂ at a concentration of less than or equal to 10 mM. In an aspect, a solution comprises MgCl₂ at a concentration of less than or equal to 9.5 mM. In an aspect, a solution comprises MgCl₂ at a concentration of less than or equal to 9 mM. In an aspect, a solution comprises MgCl₂ at a concentration of less than or equal to 8.5 mM. In an aspect, a solution comprises MgCl₂ at a concentration of less than or equal to 8 mM. In an aspect, a solution comprises MgCl₂ at a concentration of less than or equal to 7.5 mM. In an aspect, a solution comprises MgCl₂ at a concentration of less than or equal to 7 mM. In an aspect, a solution comprises MgCl₂ at a concentration of less than or equal to 6.5 mM. In an aspect, a solution comprises MgCl₂ at a concentration of less than or equal to 6 mM. In an aspect, a solution comprises MgCl₂ at a concentration of less than or equal to 5.5 mM. In another aspect, a solution comprises MgCl₂ at a concentration of less than or equal to 5 mM. In another aspect, a solution comprises MgCl₂ at a concentration of less than or equal to 4.5 mM. In an aspect, a solution comprises MgCl₂ at a concentration of less than or equal to 4 mM. In an aspect, a solution comprises MgCl₂ at a concentration of less than or equal to 3.5 mM. In an aspect, a solution comprises MgCl₂ at a concentration of less than or equal to 3 mM. In another aspect, a solution comprises MgCl₂ at a concentration of less than or equal to 2.5 mM. In an aspect, a solution comprises MgCl₂ at a concentration of less than or equal to 2 mM. In an aspect, a solution comprises MgCl₂ at a concentration of less than or equal to 1.5 mM. In another aspect, a solution comprises MgCl₂ at a concentration of less than or equal to 1 mM. In another aspect, a solution comprises MgCl₂ at a concentration of less than or equal to 0.5 mM. In another aspect, a solution comprises MgCl₂ at a concentration of less than or equal to 0.2 mM. In another aspect, a solution comprises MgCl₂ at a concentration of less than or equal to 0.1 mM. In another aspect, a solution comprises MgCl₂ at a concentration of less than or equal to 0.05 mM. In another aspect, a solution comprises MgCl₂ at a concentration of less than or equal to 0.02 mM. In another aspect, a solution comprises MgCl₂ at a concentration of less than or equal to 0.01 mM. In another aspect, a solution comprises MgCl₂ at a concentration of less than or equal to 0.005 mM. In another aspect, a solution comprises MgCl₂ at a concentration of less than or equal to 0.001 mM. In another aspect, a solution comprises MgCl₂ at a concentration of less than or equal to 0.0005 mM. In another aspect, a solution comprises MgCl₂ at a concentration of less than or equal to 0.0001 mM. In another aspect, a solution comprises MgCl₂ at a concentration of less than or equal to 0.00005 mM. In another aspect, a solution comprises MgCl₂ at a concentration of less than or equal to 0.00001 mM. In another aspect, a solution does not comprise MgCl₂.

In an aspect, a solution comprises MgCl₂ at a concentration of between 0.00001 mM and 10 mM. In an aspect, a solution comprises MgCl₂ at a concentration of between 0.00001 mM and 5 mM. In another aspect, a solution comprises MgCl₂ at a concentration of between 0.0001 mM and 10 mM. In another aspect, a solution comprises MgCl₂ at a concentration of between 0.0001 mM and 5 mM. In another aspect, a solution comprises MgCl₂ at a concentration of between 0.001 mM and 10 mM. In another aspect, a solution comprises MgCl₂ at a concentration of between 0.001 mM and 5 mM. In another aspect, a solution comprises MgCl₂ at a concentration of between 0.01 mM and 10 mM. In another aspect, a solution comprises MgCl₂ at a concentration of between 0.01 mM and 5 mM. In another aspect, a solution comprises MgCl₂ at a concentration of between 0.1 mM and 10 mM. In another aspect, a solution comprises MgCl₂ at a concentration of between 0.1 mM and 5 mM. In another aspect, a solution comprises MgCl₂ at a concentration of between 1 mM and 10 mM. In another aspect, a solution comprises MgCl₂ at a concentration of between 5 mM and 10 mM.

In an aspect, a control solution comprises MgCl₂ at a concentration of equal to or greater than 5 mM. In another aspect, a control solution comprises MgCl₂ at a concentration of equal to or greater than 7.5 mM. In another aspect, a control solution comprises MgCl₂ at a concentration of equal to or greater than 10 mM. In another aspect, a control solution comprises MgCl₂ at a concentration of equal to or greater than 12.5 mM. In another aspect, a control solution comprises MgCl₂ at a concentration of equal to or greater than 15 mM. In another aspect, a control solution comprises MgCl₂ at a concentration of equal to or greater than 17.5 mM. In another aspect, a control solution comprises MgCl₂ at a concentration of equal to or greater than 20 mM.

In an aspect, a solution comprises Mg²⁺ at a concentration of less than or equal to 10 mM. In an aspect, a solution comprises Mg²⁺ at a concentration of less than or equal to 7.5 mM. In another aspect, a solution comprises Mg²⁺ at a concentration of less than or equal to 5 mM. In another aspect, a solution comprises Mg²⁺ at a concentration of less than or equal to 5 mM. In another aspect, a solution comprises Mg²⁺ at a concentration of less than or equal to 2.5 mM. In another aspect, a solution comprises Mg²⁺ at a concentration of less than or equal to 1 mM. In another aspect, a solution comprises Mg²⁺ at a concentration of less than or equal to 0.5 mM. In another aspect, a solution comprises Mg²⁺ at a concentration of less than or equal to 0.2 mM. In another aspect, a solution comprises Mg²⁺ at a concentration of less than or equal to 0.1 mM. In another aspect, a solution comprises Mg²⁺ at a concentration of less than or equal to 0.05 mM. In another aspect, a solution comprises Mg²⁺ at a concentration of less than or equal to 0.02 mM. In another aspect, a solution comprises Mg²⁺ at a concentration of less than or equal to 0.01 mM. In another aspect, a solution comprises Mg²⁺ at a concentration of less than or equal to 0.005 mM. In another aspect, a solution comprises Mg²⁺ at a concentration of less than or equal to 0.001 mM. In another aspect, a solution comprises Mg²⁺ at a concentration of less than or equal to 0.0005 mM. In another aspect, a solution comprises Mg²⁺ at a concentration of less than or equal to 0.0001 mM. In another aspect, a solution comprises Mg²⁺ at a concentration of less than or equal to 0.00005 mM. In another aspect, a solution comprises Mg²⁺ at a concentration of less than or equal to 0.00001 mM. In another aspect, a solution does not comprise Mg²⁺.

In an aspect, a solution comprises Mg²⁺ at a concentration of between 0.00001 mM and 10 mM. In an aspect, a solution comprises Mg²⁺ at a concentration of between 0.00001 mM and 5 mM. In another aspect, a solution comprises Mg²⁺ at a concentration of between 0.0001 mM and 10 mM. In another aspect, a solution comprises Mg²⁺ at a concentration of between 0.0001 mM and 5 mM. In another aspect, a solution comprises Mg²⁺ at a concentration of between 0.001 mM and 10 mM. In another aspect, a solution comprises Mg²⁺ at a concentration of between 0.001 mM and 5 mM. In another aspect, a solution comprises Mg²⁺ at a concentration of between 0.01 mM and 10 mM. In another aspect, a solution comprises Mg²⁺ at a concentration of between 0.01 mM and 5 mM. In another aspect, a solution comprises Mg²⁺ at a concentration of between 0.1 mM and 10 mM. In another aspect, a solution comprises Mg²⁺ at a concentration of between 0.1 mM and 5 mM. In another aspect, a solution comprises Mg²⁺ at a concentration of between 1 mM and 10 mM. In another aspect, a solution comprises Mg²⁺ at a concentration of between 5 mM and 10 mM.

In an aspect, a control solution comprises Mg²⁺ at a concentration of equal to or greater than 5 mM. In another aspect, a control solution comprises Mg²⁺ at a concentration of equal to or greater than 7.5 mM. In another aspect, a control solution comprises Mg²⁺ at a concentration of equal to or greater than 10 mM. In another aspect, a control solution comprises Mg²⁺ at a concentration of equal to or greater than 12.5 mM. In another aspect, a control solution comprises Mg²⁺ at a concentration of equal to or greater than 15 mM. In another aspect, a control solution comprises Mg²⁺ at a concentration of equal to or greater than 17.5 mM. In another aspect, a control solution comprises Mg²⁺ at a concentration of equal to or greater than 20 mM.

In an aspect, a solution is provided in vivo. In another aspect, a solution is provided in vitro. In a further aspect, a solution is provided ex vivo. In an aspect, a solution is provided to a cell. In another aspect, a solution is within a cell.

In an aspect, a control solution is provided in vivo. In another aspect, a control solution is provided in vitro. In a further aspect, a control solution is provided ex vivo. In an aspect, a control solution is provided to a cell. In another aspect, a control solution is within a cell.

In an aspect, an RNA-guided CRISPR nuclease cleaves dsDNA in a solution provided herein. In another aspect, an RNA-guided CRISPR nuclease cleaves ssDNA in a solution provided herein. In an aspect, an RNA-guided CRISPR nuclease cleaves dsDNA, but not ssDNA, in a solution provided herein. In another aspect, an RNA-guided CRISPR nuclease cleaves ssDNA at a reduced rate in a solution provided herein as compared to the ssDNA cleavage rate of the RNA-guided CRISPR nuclease in a control solution.

### EDTA

EDTA (ethylene-diamine-tetraacetic acid) is a chelating agent that is known to sequester divalent and trivalent metal ions such as calcium and magnesium. This ability prevents DNA and RNA degradation as metal-dependent enzymes acting as nucleases become deactivated.

In another aspect, this disclosure provides a method of reducing ssDNA cleavage caused by an RNA-guided CRISPR nuclease comprising contacting a RNA-guided CRISPR nuclease with a target site in a solution, wherein the solution comprises EDTA at a concentration equal to greater than 0.1 mM wherein the reduced ssDNA cleavage is as compared to cleavage caused by the RNA-guided CRISPR nuclease in a control solution comprising EDTA at a concentration less than 0.1 mM. In another aspect the solution comprises EDTA at a concentration equal to or greater than 0.1 mM and MgCl2 at a concentration equal to or greater than 10 mM.

In an aspect, a solution comprises EDTA at a concentration equal to or greater than 0.1 mM. In an aspect, a solution comprises EDTA at a concentration equal to or greater than 1 mM. In an aspect, a solution comprises EDTA at a concentration equal to or greater than 5 mM. In an aspect, a solution comprises EDTA at a concentration equal to or greater than 10 mM. In an aspect, a solution comprises EDTA at a concentration equal to or greater than 15 mM. In an aspect, a solution comprises EDTA at a concentration equal to or greater than 20 mM.

### Cells

In an aspect, an engineered RNA-guided CRISPR nuclease cleaves a dsDNA molecule in a cell. In another aspect, an engineered RNA-guided CRISPR nuclease cleaves a ssDNA molecule in a cell. In an aspect, an engineered RNA-guided CRISPR nuclease cleaves a dsDNA molecule in a prokaryotic cell. In another aspect, an engineered RNA-guided CRISPR nuclease cleaves a ssDNA molecule in a prokaryotic cell. In an aspect, an engineered RNA-guided CRISPR nuclease cleaves a dsDNA molecule in a eukaryotic cell. In another aspect, an engineered RNA-guided CRISPR nuclease cleaves a ssDNA molecule in a eukaryotic cell.

In an aspect, a target nucleic acid is within a cell. In another aspect, a target nucleic acid is within a prokaryotic cell. In an aspect, a target nucleic acid is within a eukaryotic cell.

In an aspect, a prokaryotic cell is a cell from a phylum selected from the group consisting of prokaryotic cell is a cell from a phylum selected from the group consisting of Acidobacteria, Actinobacteria, Aquificae, Armatimonadetes, Bacteroidetes, Caldiserica, Chlamydie, Chlorobi, Chloroflexi, Chrysiogenetes, Coprothermobacterota, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospirae, Planctomycetes, Proteobacteria, Spirochaetes, Synergistetes, Tenericutes, Thermodesulfobacteria, Thermotogae, and Verrucomicrobia. In another aspect, a prokaryotic cell is an Escherichia coli cell. In another aspect, a prokaryotic cell is selected from a genus selected from the group consisting of Escherichia, Agrobacterium, Rhizobium, Sinorhizobium, and Staphylococcus.

In an aspect, a eukaryotic cell is an ex vivo cell. In another aspect, a eukaryotic cell is a plant cell. In another aspect, a eukaryotic cell is a plant cell in culture. In another aspect, a eukaryotic cell is an angiosperm plant cell. In another aspect, a eukaryotic cell is a gymnosperm plant cell. In another aspect, a eukaryotic cell is a monocotyledonous plant cell. In another aspect, a eukaryotic cell is a dicotyledonous plant cell. In another aspect, a eukaryotic cell is a corn cell. In another aspect, a eukaryotic cell is a rice cell. In another aspect, a eukaryotic cell is a sorghum cell. In another aspect, a eukaryotic cell is a wheat cell. In another aspect, a eukaryotic cell is a canola cell. In another aspect, a eukaryotic cell is an alfalfa cell. In another aspect, a eukaryotic cell is a soybean cell. In another aspect, a eukaryotic cell is a cotton cell. In another aspect, a eukaryotic cell is a tomato cell. In another aspect, a eukaryotic cell is a potato cell. In a further aspect, a eukaryotic cell is a cucumber cell. In another aspect, a eukaryotic cell is a millet cell. In another aspect, a eukaryotic cell is a barley cell. In another aspect, a eukaryotic cell is a Brassica cell. In another aspect, a eukaryotic cell is a grass cell. In another aspect, a eukaryotic cell is a Setaria cell. In another aspect, a eukaryotic cell is an Arabidopsis cell. In a further aspect, a eukaryotic cell is an algae cell.

In one aspect, a plant cell is an epidermal cell. In another aspect, a plant cell is a stomata cell. In another aspect, a plant cell is a trichome cell. In another aspect, a plant cell is a root cell. In another aspect, a plant cell is a leaf cell. In another aspect, a plant cell is a callus cell. In another aspect, a plant cell is a protoplast cell. In another aspect, a plant cell is a pollen cell. In another aspect, a plant cell is an ovary cell. In another aspect, a plant cell is a floral cell. In another aspect, a plant cell is a meristematic cell. In another aspect, a plant cell is an endosperm cell. In another aspect, a plant cell does not comprise reproductive material and does not mediate the natural reproduction of the plant. In another aspect, a plant cell is a somatic plant cell.

Additional provided plant cells, tissues and organs can be from seed, fruit, leaf, cotyledon, hypocotyl, meristem, embryos, endosperm, root, shoot, stem, pod, flower, inflorescence, stalk, pedicel, style, stigma, receptacle, petal, sepal, pollen, anther, filament, ovary, ovule, pericarp, phloem, and vascular tissue.

In a further aspect, a eukaryotic cell is an animal cell. In another aspect, a eukaryotic cell is an animal cell in culture. In a further aspect, a eukaryotic cell is a human cell. In another aspect, a eukaryotic cell is not a human stem cell. In a further aspect, a eukaryotic cell is a human cell in culture. In a further aspect, a eukaryotic cell is a somatic human cell. In a further aspect, a eukaryotic cell is a cancer cell. In a further aspect, a eukaryotic cell is a mammal cell. In a further aspect, a eukaryotic cell is a mouse cell. In a further aspect, a eukaryotic cell is a pig cell. In a further aspect, a eukaryotic cell is a bovid cell. In a further aspect, a eukaryotic cell is a bird cell. In a further aspect, a eukaryotic cell is a reptile cell. In a further aspect, a eukaryotic cell is an amphibian cell. In a further aspect, a eukaryotic cell is an insect cell. In a further aspect, a eukaryotic cell is an arthropod cell. In a further aspect, a eukaryotic cell is a cephalopod cell. In a further aspect, a eukaryotic cell is an arachnid cell. In a further aspect, a eukaryotic cell is a mollusk cell. In a further aspect, a eukaryotic cell is a nematode cell. In a further aspect, a eukaryotic cell is a fish cell.

In another aspect, a eukaryotic cell is a protozoan cell. In another aspect, a eukaryotic cell is a fungal cell. In an aspect, a fungal cell is a yeast cell. In an aspect, a yeast cell is a Schizosaccharomyces pombe cell. In another aspect, a yeast cell is a Saccharomyces cerevisiae cell.

### Guide Nucleic Acids

In an aspect, a method or composition provided herein comprises at least one guide nucleic acid or a nucleic acid encoding the at least one guide nucleic acid, where the at least one guide nucleic acid forms a complex with an engineered RNA-guided CRISPR nuclease, and where the at least one guide nucleic acid hybridizes with the target nucleic acid molecule. In another aspect, a ribonucleoprotein provided herein comprises an engineered RNA-guided CRISPR nuclease and at least one guide nucleic acid. In another aspect, a ribonucleoprotein provided herein comprises an RNA-guided CRISPR nuclease and at least one guide nucleic acid.

As used herein, a “guide nucleic acid” refers to a nucleic acid that forms a complex with a nuclease and then guides the complex to a specific sequence in a target nucleic acid molecule, where the guide nucleic acid and the target nucleic acid molecule share complementary sequences.

In an aspect, a guide nucleic acid comprises DNA. In another aspect, a guide nucleic acid comprises RNA. When a guide nucleic acid comprises RNA, it can be referred to as a “guide RNA.” In another aspect, a guide nucleic acid comprises DNA and RNA. In another aspect, a guide nucleic acid is single-stranded. In another aspect, a guide nucleic acid is double-stranded. In a further aspect, a guide nucleic acid is partially double-stranded.

In another aspect, a ribonucleoprotein provided herein comprises an engineered RNA-guided CRISPR nuclease and at least one guide RNA. In another aspect, a ribonucleoprotein provided herein comprises an RNA-guided CRISPR nuclease and at least one guide RNA.

In another aspect, a guide nucleic acid comprises at least 10 nucleotides. In another aspect, a guide nucleic acid comprises at least 11 nucleotides. In another aspect, a guide nucleic acid comprises at least 12 nucleotides. In another aspect, a guide nucleic acid comprises at least 13 nucleotides. In another aspect, a guide nucleic acid comprises at least 14 nucleotides. In another aspect, a guide nucleic acid comprises at least 15 nucleotides. In another aspect, a guide nucleic acid comprises at least 16 nucleotides. In another aspect, a guide nucleic acid comprises at least 17 nucleotides. In another aspect, a guide nucleic acid comprises at least 18 nucleotides. In another aspect, a guide nucleic acid comprises at least 19 nucleotides. In another aspect, a guide nucleic acid comprises at least 20 nucleotides. In another aspect, a guide nucleic acid comprises at least 21 nucleotides. In another aspect, a guide nucleic acid comprises at least 22 nucleotides. In another aspect, a guide nucleic acid comprises at least 23 nucleotides. In another aspect, a guide nucleic acid comprises at least 24 nucleotides. In another aspect, a guide nucleic acid comprises at least 25 nucleotides. In another aspect, a guide nucleic acid comprises at least 26 nucleotides. In another aspect, a guide nucleic acid comprises at least 27 nucleotides. In another aspect, a guide nucleic acid comprises at least 28 nucleotides. In another aspect, a guide nucleic acid comprises at least 30 nucleotides. In another aspect, a guide nucleic acid comprises at least 35 nucleotides. In another aspect, a guide nucleic acid comprises at least 40 nucleotides. In another aspect, a guide nucleic acid comprises at least 45 nucleotides. In another aspect, a guide nucleic acid comprises at least 50 nucleotides. In another aspect, a guide nucleic acid comprises between 10 nucleotides and 50 nucleotides. In another aspect, a guide nucleic acid comprises between 10 nucleotides and 40 nucleotides. In another aspect, a guide nucleic acid comprises between 10 nucleotides and 30 nucleotides. In another aspect, a guide nucleic acid comprises between 10 nucleotides and 20 nucleotides. In another aspect, a guide nucleic acid comprises between 16 nucleotides and 28 nucleotides. In another aspect, a guide nucleic acid comprises between 16 nucleotides and 25 nucleotides. In another aspect, a guide nucleic acid comprises between 16 nucleotides and 20 nucleotides.

In an aspect, a guide nucleic acid comprises at least 70% sequence complementarity to a target nucleic acid sequence. In an aspect, a guide nucleic acid comprises at least 75% sequence complementarity to a target nucleic acid sequence. In an aspect, a guide nucleic acid comprises at least 80% sequence complementarity to a target nucleic acid sequence. In an aspect, a guide nucleic acid comprises at least 85% sequence complementarity to a target nucleic acid sequence. In an aspect, a guide nucleic acid comprises at least 90% sequence complementarity to a target nucleic acid sequence. In an aspect, a guide nucleic acid comprises at least 91% sequence complementarity to a target nucleic acid sequence. In an aspect, a guide nucleic acid comprises at least 92% sequence complementarity to a target nucleic acid sequence. In an aspect, a guide nucleic acid comprises at least 93% sequence complementarity to a target nucleic acid sequence. In an aspect, a guide nucleic acid comprises at least 94% sequence complementarity to a target nucleic acid sequence. In an aspect, a guide nucleic acid comprises at least 95% sequence complementarity to a target nucleic acid sequence. In an aspect, a guide nucleic acid comprises at least 96% sequence complementarity to a target nucleic acid sequence. In an aspect, a guide nucleic acid comprises at least 97% sequence complementarity to a target nucleic acid sequence. In an aspect, a guide nucleic acid comprises at least 98% sequence complementarity to a target nucleic acid sequence. In an aspect, a guide nucleic acid comprises at least 99% sequence complementarity to a target nucleic acid sequence. In an aspect, a guide nucleic acid comprises 100% sequence complementarity to a target nucleic acid sequence. In another aspect, a guide nucleic acid comprises between 70% and 100% sequence complementarity to a target nucleic acid sequence. In another aspect, a guide nucleic acid comprises between 80% and 100% sequence complementarity to a target nucleic acid sequence. In another aspect, a guide nucleic acid comprises between 90% and 100% sequence complementarity to a target nucleic acid sequence.

Some RNA-guided CRISPR nucleases, such as CasX and Cas9, require another non-coding RNA component, referred to as a trans-activating crRNA (tracrRNA), to have functional activity. Guide nucleic acid molecules provided herein can combine a crRNA and a tracrRNA into one nucleic acid molecule in what is herein referred to as a “single guide RNA” (sgRNA). The gRNA guides the active CasX complex to a target site, where CasX can cleave the target site.

In an aspect, a guide nucleic acid comprises a crRNA. In another aspect, a guide nucleic acid comprises a tracrRNA. In a further aspect, a guide nucleic acid comprises an sgRNA.

In an aspect, a guide nucleic acid provided herein can be expressed from a recombinant vector in vivo. In an aspect, a guide nucleic acid provided herein can be expressed from a recombinant vector in vitro. In an aspect, a guide nucleic acid provided herein can be expressed from a recombinant vector ex vivo. In an aspect, a guide nucleic acid provided herein can be expressed from a nucleic acid molecule in vivo. In an aspect, a guide nucleic acid provided herein can be expressed from a nucleic acid molecule in vitro. In an aspect, a guide nucleic acid provided herein can be expressed from a nucleic acid molecule ex vivo. In another aspect, a guide nucleic acid provided herein can be synthetically synthesized.

### Target Nucleic Acids

In an aspect, a dsRNA molecule comprises a target nucleic acid. In another aspect, a dsRNA molecule comprises a target region.

As used herein, a “target nucleic acid” or “target nucleic acid molecule” or “target nucleic acid sequence” refers to a selected nucleic acid molecule or a selected sequence or region of a nucleic acid molecule in which a modification (e.g., cleavage) is desired. Similarly, a “target dsRNA” refers to a selected double-stranded DNA molecule in which a modification (e.g., cleavage) is desired.

As used herein, a “target region” or “targeted region” refers to the portion of a target nucleic acid that is cleaved by an engineered RNA-guided CRISPR nuclease. In contrast to a non-target nucleic acid (e.g., non-target ssDNA) or non-target region, a target region comprises significant complementarity to a guide nucleic acid or a guide RNA. In an aspect, a target region is 100% complementary to a guide nucleic acid. In another aspect, a target region is 99% complementary to a guide nucleic acid. In another aspect, a target region is 98% complementary to a guide nucleic acid. In another aspect, a target region is 97% complementary to a guide nucleic acid. In another aspect, a target region is 96% complementary to a guide nucleic acid. In another aspect, a target region is 95% complementary to a guide nucleic acid. In another aspect, a target region is 94% complementary to a guide nucleic acid. In another aspect, a target region is 93% complementary to a guide nucleic acid. In another aspect, a target region is 92% complementary to a guide nucleic acid. In another aspect, a target region is 91% complementary to a guide nucleic acid. In another aspect, a target region is 90% complementary to a guide nucleic acid. In another aspect, a target region is 85% complementary to a guide nucleic acid. In another aspect, a target region is 80% complementary to a guide nucleic acid. In an aspect, a target region is adjacent to a nucleic acid sequence that is 100% complementary to a guide nucleic acid. In another aspect, a target region is adjacent to a nucleic acid sequence that is 99% complementary to a guide nucleic acid. In another aspect, a target region is adjacent to a nucleic acid sequence that is 98% complementary to a guide nucleic acid. In another aspect, a target region is adjacent to a nucleic acid sequence that is 97% complementary to a guide nucleic acid. In another aspect, a target region is adjacent to a nucleic acid sequence that is 96% complementary to a guide nucleic acid. In another aspect, a target region is adjacent to a nucleic acid sequence that is 95% complementary to a guide nucleic acid. In another aspect, a target region is adjacent to a nucleic acid sequence that is 94% complementary to a guide nucleic acid. In another aspect, a target region is adjacent to a nucleic acid sequence that is 93% complementary to a guide nucleic acid. In another aspect, a target region is adjacent to a nucleic acid sequence that is 92% complementary to a guide nucleic acid. In another aspect, a target region is adjacent to a nucleic acid sequence that is 91% complementary to a guide nucleic acid. In another aspect, a target region is adjacent to a nucleic acid sequence that is 90% complementary to a guide nucleic acid. In another aspect, a target region is adjacent to a nucleic acid sequence that is 85% complementary to a guide nucleic acid. In another aspect, a target region is adjacent to a nucleic acid sequence that is 80% complementary to a guide nucleic acid.

In an aspect, a target region comprises at least one PAM site. In an aspect, a target region is adjacent to a nucleic acid sequence that comprises at least one PAM site. In another aspect, a target region is within 5 nucleotides of at least one PAM site. In a further aspect, a target region is within 10 nucleotides of at least one PAM site. In another aspect, a target region is within 15 nucleotides of at least one PAM site. In another aspect, a target region is within 20 nucleotides of at least one PAM site. In another aspect, a target region is within 25 nucleotides of at least one PAM site. In another aspect, a target region is within 30 nucleotides of at least one PAM site.

In an aspect, a target nucleic acid comprises RNA. In another aspect, a target nucleic acid comprises DNA. In an aspect, a target nucleic acid is single-stranded. In another aspect, a target nucleic acid is double-stranded. In an aspect, a target nucleic acid comprises single-stranded RNA. In an aspect, a target nucleic acid comprises ssDNA. In an aspect, a target nucleic acid comprises double-stranded RNA. In an aspect, a target nucleic acid comprises dsDNA. In an aspect, a target nucleic acid comprises genomic DNA. In an aspect, a target nucleic acid is positioned within a nuclear genome. In an aspect, a target nucleic acid comprises chromosomal DNA. In an aspect, a target nucleic acid comprises plasmid DNA. In an aspect, a target nucleic acid is positioned within a plasmid. In an aspect, a target nucleic acid comprises mitochondrial DNA. In an aspect, a target nucleic acid is positioned within a mitochondrial genome. In an aspect, a target nucleic acid comprises plastid DNA. In an aspect, a target nucleic acid is positioned within a plastid genome. In an aspect, a target nucleic acid comprises chloroplast DNA. In an aspect, a target nucleic acid is positioned within a chloroplast genome. In an aspect, a target nucleic acid is positioned within a genome selected from the group consisting of a nuclear genome, a mitochondrial genome, and a plastid genome.

In an aspect, a target nucleic acid encodes a gene. As used herein, a “gene” refers to a polynucleotide that can produce a functional unit (e.g., without being limiting, for example, a protein, or a non-coding RNA molecule). A gene can comprise a promoter, an enhancer sequence, a leader sequence, a transcriptional start site, a transcriptional stop site, a polyadenylation site, one or more exons, one or more introns, a 5′-UTR, a 3′-UTR, or any combination thereof. A “gene sequence” can comprise a polynucleotide sequence encoding a promoter, an enhancer sequence, a leader sequence, a transcriptional start site, a transcriptional stop site, a polyadenylation site, one or more exons, one or more introns, a 5′-UTR, a 3′-UTR, or any combination thereof. In one aspect, a gene encodes a non-protein-coding RNA molecule or a precursor thereof. In another aspect, a gene encodes a protein. In some embodiments, the target nucleic acid is selected from the group consisting of: a promoter, an enhancer sequence, a leader sequence, a transcriptional start site, a transcriptional stop site, a polyadenylation site, an exon, an intron, a splice site, a 5′-UTR, a 3′-UTR, a protein coding sequence, a non-protein-coding sequence, a miRNA, a pre-miRNA and a miRNA binding site.

Non-limiting examples of a non-protein-coding RNA molecule include a microRNA (miRNA), a miRNA precursor (pre-miRNA), a small interfering RNA (siRNA), a small RNA (18-26 nt in length) and precursor encoding same, a heterochromatic siRNA (hc-siRNA), a Piwi-interacting RNA (piRNA), a hairpin double strand RNA (hairpin dsRNA), a trans-acting siRNA (ta-siRNA), a naturally occurring antisense siRNA (nat-siRNA), a CRISPR RNA (crRNA), a tracer RNA (tracrRNA), a guide RNA (gRNA), and a single guide RNA (sgRNA).

### Nucleic Acids and Polypeptides

The use of the term “polynucleotide” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise deoxyribonucleotides, ribonucleotides, or combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In another aspect, a nucleic acid molecule provided herein is an RNA molecule. In an aspect, a nucleic acid molecule provided herein is single-stranded. In another aspect, a nucleic acid molecule provided herein is double-stranded.

In one aspect, methods and compositions provided herein comprise a vector. As used herein, the terms “vector” or “plasmid” are used interchangeably and refer to a circular, double-stranded DNA molecule that is physically separate from chromosomal DNA. In one aspect, a plasmid or vector used herein is capable of replication in vivo. In another aspect, a nucleic acid encoding a catalytically inactive guided-nuclease is provided in a vector. In a further aspect, a nucleic acid encoding a guide nucleic acid is provided in a vector. In still yet another aspect, a nucleic acid encoding a catalytically inactive guided-nuclease and a nucleic acid encoding a guide nucleic acid are provided in a single vector.

In an aspect, this disclosure provides a polynucleotide encoding an engineered RNA-guided CRISPR nuclease. In another aspect, a vector comprises a polynucleotide encoding an engineered RNA-guided CRISPR nuclease. In an aspect, this disclosure provides a polynucleotide encoding an RNA-guided CRISPR nuclease. In another aspect, a vector comprises a polynucleotide encoding an RNA-guided CRISPR nuclease. In an aspect, this disclosure provides a polynucleotide encoding a guide nucleic acid. In another aspect, this disclosure provides a vector encoding a guide nucleic acid.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Without being limiting, nucleic acids can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, N.Y.).

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

The terms “percent identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

The terms “percent sequence complementarity” or “percent complementarity” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

For optimal alignment of sequences to calculate their percent identity, various pair-wise or multiple sequence alignment algorithms and programs are known in the art, such as ClustalW or Basic Local Alignment Search Tool (BLAST®), etc., that can be used to compare the sequence identity or similarity between two or more nucleotide or protein sequences. Although other alignment and comparison methods are known in the art, the alignment and percent identity between two sequences (including the percent identity ranges described above) can be as determined by the ClustalW algorithm, see, e.g., Chenna R. et al., “Multiple sequence alignment with the Clustal series of programs,” Nucleic Acids Research 31: 3497-3500 (2003); Thompson J D et al., “Clustal W: Improving the sensitivity of progressive multiple sequence alignment through sequence weighting, position-specific gap penalties and weight matrix choice,” Nucleic Acids Research 22: 4673-4680 (1994); Larkin M A et al., “Clustal W and Clustal X version 2.0,” Bioinformatics 23: 2947-48 (2007); and Altschul, S. F., Gish, W., Miller, W., Myers, E. W. & Lipman, D. J. (1990) “Basic local alignment search tool.” J. Mol. Biol. 215:403-410 (1990), the entire contents and disclosures of which are incorporated herein by reference.

As used herein, a first nucleic acid molecule can “hybridize” a second nucleic acid molecule via non-covalent interactions (e.g., Watson-Crick base-pairing) in a sequence-specific, antiparallel manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine pairing with thymine, adenine pairing with uracil, and guanine (G) pairing with cytosine (C) [DNA, RNA]. In addition, it is also known in the art that for hybridization between two RNA molecules (e.g., dsRNA), guanine base pairs with uracil. For example, G/U base-pairing is partially responsible for the degeneracy (i.e., redundancy) of the genetic code in the context of tRNA anti-codon base-pairing with codons in mRNA. In the context of this disclosure, a guanine of a protein-binding segment (dsRNA duplex) of a subject DNA-targeting RNA molecule is considered complementary to an uracil, and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or fewer nucleotides) the position of mismatches becomes important (see Sambrook et al.). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST® programs (basic local alignment search tools) and PowerBLAST programs known in the art (see Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

### Transformation/Transfection

Any method provided herein can involve transient transfection or stable transformation of a cell of interest (e.g., a eukaryotic cell, a prokaryotic cell). In an aspect, a nucleic acid molecule encoding an engineered RNA-guided CRISPR nuclease is stably transformed. In another aspect, a nucleic acid molecule encoding an engineered RNA-guided CRISPR nuclease is transiently transfected. In an aspect, a nucleic acid molecule encoding an RNA-guided CRISPR nuclease is stably transformed. In another aspect, a nucleic acid molecule encoding an RNA-guided CRISPR nuclease is transiently transfected. In an aspect, a nucleic acid molecule encoding a guide nucleic acid is stably transformed. In another aspect, a nucleic acid molecule encoding a guide nucleic acid is transiently transfected.

Numerous methods for transforming cells with a recombinant nucleic acid molecule or construct are known in the art, which can be used according to methods of the present application. Any suitable method or technique for transformation of a cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants.

In an aspect, a method comprises providing a cell with an engineered RNA-guided CRISPR nuclease, or a nucleic acid encoding the engineered RNA-guided CRISPR nuclease, via Agrobacterium-mediated transformation. In an aspect, a method comprises providing a cell with an engineered RNA-guided CRISPR nuclease, or a nucleic acid encoding the engineered RNA-guided CRISPR nuclease, via polyethylene glycol-mediated transformation. In an aspect, a method comprises providing a cell with an engineered RNA-guided CRISPR nuclease, or a nucleic acid encoding the engineered RNA-guided CRISPR nuclease, via biolistic transformation. In an aspect, a method comprises providing a cell with an engineered RNA-guided CRISPR nuclease, or a nucleic acid encoding the engineered RNA-guided CRISPR nuclease, via liposome-mediated transfection. In an aspect, a method comprises providing a cell with an engineered RNA-guided CRISPR nuclease, or a nucleic acid encoding the engineered RNA-guided CRISPR nuclease, via viral transduction. In an aspect, a method comprises providing a cell with an engineered RNA-guided CRISPR nuclease, or a nucleic acid encoding the engineered RNA-guided CRISPR nuclease, via use of one or more delivery particles. In an aspect, a method comprises providing a cell with an engineered RNA-guided CRISPR nuclease, or a nucleic acid encoding the engineered RNA-guided CRISPR nuclease, via microinjection. In an aspect, a method comprises providing a cell with an engineered RNA-guided CRISPR nuclease, or a nucleic acid encoding the engineered RNA-guided CRISPR nuclease, via electroporation.

In an aspect, a method comprises providing a cell with a guide nucleic acid, or a nucleic acid encoding the guide nucleic acid, via Agrobacterium-mediated transformation. In an aspect, a method comprises providing a cell with a guide nucleic acid, or a nucleic acid encoding the guide nucleic acid, via polyethylene glycol-mediated transformation. In an aspect, a method comprises providing a cell with a guide nucleic acid, or a nucleic acid encoding the guide nucleic acid, via biolistic transformation. In an aspect, a method comprises providing a cell with a guide nucleic acid, or a nucleic acid encoding the guide nucleic acid, via liposome-mediated transfection. In an aspect, a method comprises providing a cell with a guide nucleic acid, or a nucleic acid encoding the guide nucleic acid, via viral transduction. In an aspect, a method comprises providing a cell with a guide nucleic acid, or a nucleic acid encoding the guide nucleic acid, via use of one or more delivery particles. In an aspect, a method comprises providing a cell with a guide nucleic acid, or a nucleic acid encoding the guide nucleic acid, via microinjection. In an aspect, a method comprises providing a cell with a guide nucleic acid, or a nucleic acid encoding the guide nucleic acid, via electroporation.

In an aspect, a ribonucleoprotein is provided to a cell via a method selected from the group consisting of Agrobacterium-mediated transformation, polyethylene glycol-mediated transformation, biolistic transformation, liposome-mediated transfection, viral transduction, the use of one or more delivery particles, microinjection, and electroporation.

Other methods for transformation, such as vacuum infiltration, pressure, sonication, and silicon carbide fiber agitation, are also known in the art and envisioned for use with any method provided herein.

Methods of transforming cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any appropriate method known to those skilled in the art can be used to transform a plant cell with any of the nucleic acid molecules provided herein.

Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

Delivery vehicles, vectors, particles, nanoparticles, formulations and components thereof for expression of one or more elements of a nucleic acid molecule or a protein are as used in WO 2014/093622 (PCT/US2013/074667). In an aspect, a method of providing a nucleic acid molecule or a protein to a cell comprises delivery via a delivery particle. In an aspect, a method of providing a nucleic acid molecule or a protein to a cell comprises delivery via a delivery vesicle. In an aspect, a delivery vesicle is selected from the group consisting of an exosome and a liposome. In an aspect, a method of providing a nucleic acid molecule or a protein to a cell comprises delivery via a viral vector. In an aspect, a viral vector is selected from the group consisting of an adenovirus vector, a lentivirus vector, and an adeno-associated viral vector. In another aspect, a method providing a nucleic acid molecule or a protein to a cell comprises delivery via a nanoparticle. In an aspect, a method providing a nucleic acid molecule or a protein to a cell comprises microinjection. In an aspect, a method providing a nucleic acid molecule or a protein to a cell comprises polycations. In an aspect, a method providing a nucleic acid molecule or a protein to a cell comprises a cationic oligopeptide.

In an aspect, a delivery particle is selected from the group consisting of an exosome, an adenovirus vector, a lentivirus vector, an adeno-associated viral vector, a nanoparticle, a polycation, and a cationic oligopeptide. In an aspect, a method provided herein comprises the use of one or more delivery particles. In another aspect, a method provided herein comprises the use of two or more delivery particles. In another aspect, a method provided herein comprises the use of three or more delivery particles.

Suitable agents to facilitate transfer of proteins; nucleic acids, mutagens and ribonucleoproteins into a plant cell include agents that increase permeability of the exterior of the plant or that increase permeability of plant cells to oligonucleotides, polynucleotides, proteins, or ribonucleoproteins. Such agents to facilitate transfer of the composition into a plant cell include a chemical agent, or a physical agent, or combinations thereof. Chemical agents for conditioning includes (a) surfactants, (b) an organic solvents or an aqueous solutions or aqueous mixtures of organic solvents. (c) oxidizing agents, (e) acids, (f) bases, (g) oils (h) enzymes, or combinations thereof.

Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions). Naturally derived or synthetic oils with or without surfactants or emulsifiers can be used, e.g. plant-sourced, oils, crop oils (such as those listed in the 9^{th} Compendium of Herbicide Adjuvants, publicly available on line at www.herbicide.adjuvants.com) can be used, e.g., paraffinic oils, polyol fatty acid esters, or oils with short-chain molecules modified with amides or polyamines such as polyethyleneimine or N-pyrrolidine.

Examples of useful surfactants include sodium or lithium salts of fatty acids (such as tallow or tallowamines or phospholipids) and organosilicone surfactants. Other useful surfactants include organosilicone surfactants including nonionic organosilicone surfactants, e.g., trisiloxane ethoxylate surfactants or a silicone polyether copolymer such as a copolymer of polyalkylene oxide modified heptamethyl trisiloxane and allyloxypolypropylene glycol methyl ether (commercially available as Silwet® L-77).

Useful physical agents can include (a) abrasives such as carborundum, corundum, sand, calcite, pumice, garnet, and the like, (b) nanoparticles such as carbon nanotubes or (c) a physical force. Carbon nanotubes are disclosed by Kam et al. (2004)/. Am. Chem. Soc, 126 (22):6850-6851, Liu et al. (2009) Nano Lett, 9(3): 1007-1010, and Khodakovskava et al. (2009) ACS Nano, 3(10):3221-3227. Physical force agents can include heating, chilling, the application of positive pressure, or ultrasound treatment. Embodiments of the method can optionally include an incubation step, a neutralization step (e.g., to neutralize an acid, base, or oxidizing agent, or to inactivate an enzyme), a rinsing step, or combinations thereof. The methods of the invention can further include the application of other agents which will have enhanced effect due to the silencing of certain genes. For example, when a polynucleotide is designed to regulate genes that provide herbicide resistance, the subsequent application of the herbicide can have a dramatic effect on herbicide efficacy.

Agents for laboratory conditioning of a plant cell to permeation by polynucleotides include, e.g., application of a chemical agent, enzymatic treatment, heating or chilling, treatment with positive or negative pressure, or ultrasound treatment. Agents for conditioning plants in a field include chemical agents such as surfactants and salts.

In an aspect, ssDNA or dsDNA is contacted by an engineered RNA-guided CRISPR nuclease in vivo. In an aspect, a ssDNA or dsDNA is contacted by an engineered RNA-guided CRISPR nuclease ex vivo. In an aspect, a ssDNA or dsDNA is contacted by an engineered RNA-guided CRISPR nuclease in vitro.

In an aspect, a target nucleic acid is contacted by a ribonucleoprotein in vivo. In an aspect, a target nucleic acid is contacted by a ribonucleoprotein ex vivo. In an aspect, a target nucleic acid is contacted by a ribonucleoprotein in vitro.

Recipient plant cell or explant targets for transformation include, but are not limited to, a seed cell, a fruit cell, a leaf cell, a cotyledon cell, a hypocotyl cell, a meristem cell, an embryo cell, an endosperm cell, a root cell, a shoot cell, a stem cell, a pod cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, a phloem cell, a bud cell, or a vascular tissue cell. In another aspect, this disclosure provides a plant chloroplast. In a further aspect, this disclosure provides an epidermal cell, a stomata cell, a trichome cell, a root hair cell, a storage root cell, or a tuber cell. In another aspect, this disclosure provides a protoplast. In another aspect, this disclosure provides a plant callus cell. Any cell from which a fertile plant can be regenerated is contemplated as a useful recipient cell for practice of this disclosure. Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making transgenic plants of this disclosure (e.g., various media and recipient target cells, transformation of immature embryos, and subsequent regeneration of fertile transgenic plants) are disclosed, for example, in U.S. Pat. Nos. 6,194,636 and 6,232,526 and U. S. Patent Application Publication 2004/0216189, all of which are incorporated herein by reference. Transformed explants, cells or tissues can be subjected to additional culturing steps, such as callus induction, selection, regeneration, etc., as known in the art. Transformed cells, tissues or explants containing a recombinant DNA insertion can be grown, developed or regenerated into transgenic plants in culture, plugs or soil according to methods known in the art. In one aspect, this disclosure provides plant cells that are not reproductive material and do not mediate the natural reproduction of the plant. In another aspect, this disclosure also provides plant cells that are reproductive material and mediate the natural reproduction of the plant. In another aspect, this disclosure provides plant cells that cannot maintain themselves via photosynthesis. In another aspect, this disclosure provides somatic plant cells. Somatic cells, contrary to germline cells, do not mediate plant reproduction. In one aspect, this disclosure provides a non-reproductive plant cell.

### EXAMPLES

### Example 1. In Vitro DNase Activity Assay

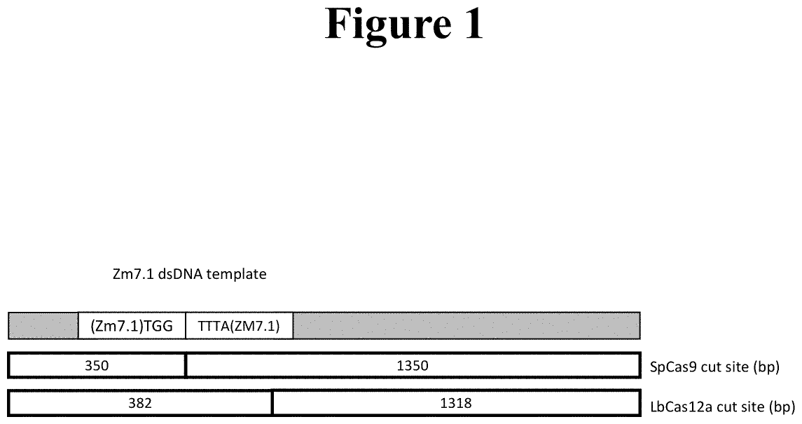

An in vitro deoxyribonuclease (DNase) activity assay was developed to investigate the single-stranded (ss) and double-stranded (ds) DNase activity of the RNA guided CRISPR nuclease LbCas12a (Lachnospiraceae bacterium ND2006 Cas12a). Two DNA substrates were utilized in this assay. The synthetic dsDNA substrate used in the assay was Zm7.1, a 1700 bp PCR product (SEQ ID NO: 1) that comprised two unique target sites. A Cas9 target site (Cas9_Zm7.1) is located 350 nucleotides into the sequence and was recognized by a Cas9 specific single guide RNA (Cas9_Zm7.1_sgRNA), the sequence of which has previously been disclosed in U.S. Patent Application Publication No. 2017/0166912, which is herein incorporated by reference in its entirety. This 1700 bp product also comprises an LbCas12a target site (Cas12a_Zm7.1) located 382 nucleotides into the sequence that is recognized by a Cas12a specific guide RNA (Cas12a-Zm7.1_gRNA) (SEQ ID NO: 21). Demonstration of dsDNA cutting activity by SpCas9 (Streptococcus pyogenes Cas9) and its cognate Cas9-zm7.1_sgRNA would result in 350 bp and 1350 bp DNA fragments. See FIG. 1. Demonstration of dsDNA cutting activity by LbCas12a and its cognate Cas12a-Zm7.1 gRNA would result in 382 bp and ˜1318 bp DNA fragments. See FIG. 1.

The ssDNA substrate used in the assay was the M13mp18 ssDNA phage sequence (New England Biolabs, #N4040s) previously described and used in Chen et al., Science, 360:436-439 (2018) April 27; 360(6387):436-439. To evaluate if dsDNA cutting and ssDNase activities of LbCas12a can be separated, these substrates were evaluated in reactions individually as well as in combined reactions.

The LbCas12a wildtype protein (SEQ ID NO: 2) and variants were expressed and purified from Escherichia coli. For this purpose, the open reading frame of LbCas12a was codon-optimized for optimal expression in E. coli cells (SEQ ID NO: 3). A histidine tag sequence (SEQ ID NO: 4) was introduced at the 5′ end of the gene. Additionally, two nuclear localization signals (NLS) (SEQ ID NOs: 5 and 6) were introduced at the 5′ and 3′ ends of LbCas12a open reading frame. Finally, a unique Sph1 site was introduced at the 3′ end of the DNA resulting in an alanine residue at the C-terminal end of the protein. The LbCas12a fusion proteins used in the in vitro DNAse assays had the following configuration: HIStag:NLS:LbCas12a:NLS.

Reactions were carried out in cleavage buffer consisting of 20 mM HEPES, 10 mM MgCl₂ and 0.5 mM DTT and comprised 26.7 nM dsDNA substrate and/or 12.54 nM M13 ssDNA substrate. Purified LbCas12a or LbCas12a variant proteins were assembled with or without the cognate gRNA (100 μM) and incubated with the dsDNA, ssDNA, or a combination of dsDNA and ssDNA. The protein amounts were adjusted to accommodate the specific protein to DNA ratio that was investigated for each reaction. The reactions were carried out at 37° C. for 45 minutes, unless otherwise stated, and quenched with proteinase K treatment at 65° C. for 15 minutes. The samples were separated and analyzed on a 1.8% TBE Agarose gel.

### Example 2. ssDNase Activity of LbCas12a

It has previously been reported that when paired with its guide RNA and in the presence of a target DNA, Cas12a exhibits non-specific single stranded (ss) DNAse activity resulting in degradation of non-target ssDNA (see, for example, Chen et al. Science, 360:436-439 (2018) April 27; 360(6387):436-439). The in vitro DNAse assay described in Example 1 was used to investigate the DNAse activity of LbCas12a. Specifically, the guide RNA directed DNA cutting activity of LbCas12a was tested on dsDNA, ssDNA, and a combination of dsDNA and ssDNA templates. The experimental set up is described in Table 1. The gRNA-directed and substrate-specific targeted dsDNA cutting activity of LbCas12a was tested in assay 4 (see Table 1). The reaction mixture was essentially as described in Experiment 1 and contained purified LbCas12a protein mixed with Zm7.1 dsDNA at a ratio of 60:1 along with Cas12a-zm7.1 gRNA. Three controls were run in parallel (see Assays 1-3, Table 1). Assay 1 comprised the Zm7.1 template dsDNA but lacked the Cas12a nuclease and gRNA. Assay 2 comprised the template and nuclease but lacked the cognate Cas12a gRNA. Assay 3 comprised the template, Cas12a nuclease, and a Cas9 guide RNA that is not expected to be recognized by Cas12a.

The non-target specific ssDNase activity of LbCas12a was tested in Assay 8, Table 1. The reaction mixture contained purified LbCas12a mixed with M13mp18 ssDNA at a ratio of 60:1 along with Cas12a-zm7.1 gRNA. Three controls were run in parallel (see Assays 5-7, Table 1) and are detailed in Table 1.

The cleavage activity of LbCas12a in the presence of a mixture of dsDNA and ssDNA templates was tested in Assay 12. See Table 1. The reaction mixture contained purified LbCas12a mixed with Zm7.1 dsDNA and ssDNA M13mp18 at a ratio of 60:1 of protein to DNA along with Cas12a-zm7.1 gRNA. 3 controls were run in parallel (Assays 9, 10, and 11) and are described in Table 1.

The reactions were carried out at 37° C. for 45 minutes and quenched with proteinase K. The samples were then separated and analyzed on a 1.8% TBE Agarose gel. As shown in Table 1, in reactions comprising Zm7.1 template DNA with LbCas12a and its cognate gRNA (Assays 4 and 12), ˜382 bp and ˜1318 bp DNA fragment bands were observed. This suggests that in the presence of the cognate Cas12a guide RNA, LbCas12a carried out sequence-specific cleavage of both strands of the ˜1700 bp Zm7.1 dsDNA to near completion to release the ˜382 bp and ˜1318 bp fragments. In reactions comprising the M13mp18 ssDNA with LbCas12a and its gRNA (Table 1, Assays 8 and 12), the M13mp18 ssDNA band was either absent or band intensity was significantly less than that seen in the controls. This suggests that in the presence of its cognate guide RNA, LbCas12a degraded the M13mp18 ssDNA, thus confirming its non-specific ssDNAse activity.

It has previously been reported that mutations in key residues within the DNA targeting domain of Cas12a protein can completely abolish the DNA cleavage activity (see, for example, Zetsche et al., Cell, 163:759, (2015)). D832 and E925 residues within LbCas12a were mutated to Alanine residues and the resultant Cas12a variant was designated dLbCas12a (Dead LbCas12a) (SEQ ID NO: 7). dLbCas12a was tested for its in vitro DNAse activity using the assay described in Example 1. The experimental details are described in Table 2. As shown in Table 2 (Assays 4 and 12), in reactions comprising Zm7.1 template DNA with dLbCas12a and its cognate gRNA, the full length ˜1700 bp Zm7.1 DNA was observed while the ˜382 bp and ˜1318 bp fragments were not observed. This suggests that at 60:1 protein to DNA ratio, dLbCas12a did not cut dsDNA in the presence of the cognate Cas12a guide RNA. As described in Table 2 (Assays 8 and 12), in reactions comprising the M13mp18 ssDNA with dLbCas12a and its gRNA, the M13 ssDNA was observed and the band intensity was comparable to the controls. This suggests that at 60:1 protein to DNA ratio, dLbCas12a did not degrade the M13mp18 ssDNA in the presence of the cognate Cas12a guide RNA.

TABLE 1
DNase activity assay with LbCas12a. For targeted dsDNA cleavage, “Yes” refers to the
observation of only the ~382 bp and ~1318 bp DNA fragments on the gel. ‘‘No” refers to the
observation of the full length ~1700 bp Zm7.1DNA and absence of the ~382 bp DNA fragment
and ~1318 bpDNA fragments. For ssDNase activity, “Yes” refers to the observation that
M13mp18 ssDNA band was either absent or its intensity was less than that observed in the
controls. “No” refers to observation that M13mp18 ssDNA band intensity was
comparable to the intensity observed in the controls.

| | | | | | | Targeted | |
|---|---|---|---|---|---|---|---|
| | | | | | | dsDNA | ssDNA |
| | | | | | | cleavage | degradation |
| Assay/ | Template | Type of | | | | (N/A = Not | (N/A = Not |
| Lane | type | assay | Template | Nuclease | gRNA | applicable) | applicable) |
| 1 | dsDNA | Control | Zm7.1 | — | — | No | N/A |
| 2 | | Control | Zm7.1 | LbCas12a | — | No | N/A |
| 3 | | Control | Zm7.1 | LbCas12a | Cas9 gRNA | No | N/A |
| 4 | | Test | Zm7.1 | LbCas12a | Cas12a gRNA | Yes | N/A |
| 5 | ssDNA | Control | M13mp18 | — | — | N/A | No |
| 6 | | Control | M13mp18 | LbCas12a | — | N/A | No |
| 7 | | Control | M13mp18 | LbCas12a | Cas9 gRNA | N/A | No |
| 8 | | Test | M13mp18 | LbCas12a | Cas12a gRNA | N/A | Yes |
| 9 | dsDNA + | Control | Zm7.1 + | — | — | No | No |
| | ssDNA | | M13mp18 | | | | |
| 10 | | Control | Zm7.1 + | LbCas12a | — | No | No |
| | | | M13mp18 | | | | |
| 11 | | Control | Zm7.1 + | LbCas12a | Cas9 gRNA | No | No |
| | | | M13mp18 | | | | |
| 12 | | Test | Zm7.1 + | LbCas12a | Cas12a gRNA | Yes | Yes |
| | | | M13mp18 | | | | |

TABLE 2
DNase activity of dLbCas12a (D832A/E925A). Targeted dsDNA cleavage: “No” refers
to the observation of only the full length ~1700 bp Zm7.1DNA and absence of the ~382 bp DNA
fragment and ~1318 bp DNA fragments. For ssDNAse activity, “No” refers to observation where
M13mp18 ssDNA band intensity is comparable to that observed in the controls.

| | | | | | | Targeted | |
|---|---|---|---|---|---|---|---|
| | | | | Nuclease | | dsDNA | ssDNA |
| | | | | (dLbCas12a = | | cleavage | degradation |
| Assay/ | Template | Type of | | D832A/E925A | | (N/A = Not | (N/A = Not |
| Lane | type | assay | Template | variant) | gRNA | applicable) | applicable) |
| 1 | dsDNA | Control | Zm7.1 | — | — | No | N/A |
| 2 | | Control | Zm7.1 | dLbCas12a | — | No | N/A |
| 3 | | Control | Zm7.1 | dLbCas12a | Cas9 gRNA | No | N/A |
| 4 | | Test | Zm7.1 | dLbCas12a | Cas12a gRNA | No | N/A |
| 5 | ssDNA | Control | M13mp18 | — | — | N/A | No |
| 6 | | Control | M13mp18 | dLbCas12a | — | N/A | No |
| 7 | | Control | M13mp18 | dLbCas12a | Cas9 gRNA | N/A | No |
| 8 | | Test | M13mp18 | dLbCas12a | Cas12a gRNA | N/A | No |
| 9 | dsDNA + | Control | Zm7.1 + | — | — | No | No |
| | ssDNA | | M13mp18 | | | | |
| 10 | | Control | Zm7.1 + | dLbCas12a | — | No | No |
| | | | M13mp18 | | | | |
| 11 | | Control | Zm7.1 + | dLbCas12a | Cas9 gRNA | No | No |
| | | | M13mp18 | | | | |
| 12 | | Test | Zm7.1 + | dLbCas12a | Cas12a gRNA | No | No |
| | | | M13mp18 | | | | |

### Example 3. Identification of an LbCas12a Variant with Reduced ssDNase Activity

DNA nuclease activity takes place at the RuvC-Nuc domain interface of the Cas12a protein (see, for example, Yamano et al., Cell 165, 4:949, (2016). Two candidate residues within this region, R1138 and E925 were mutated to alanine and the ssDNase activity of the variants was tested. The amino acid sequence of LbCas12aR1138A is set forth as SEQ ID NO: 8, and the amino acid sequence of LbCas12aE925A is set forth as SEQ ID NO: 9. Since the R1138A mutation occurs within the predicted DNA catalytic domain of Cas12a, this LbCas12a variant is predicted to be a nickase and cleave only a single strand of the target DNA (see, for example, U.S. Patent Application Publication No. 2018/0030425). The variants were investigated for their in vitro DNase activity using the assay described in Example 1. The test assay comprised the purified LbCas12a protein variant mixed with Zm7.1 dsDNA at a ratio of 60:1 along with Cas12a-zm7.1 gRNA. Three controls were run in parallel. The assays and results for LbCas12aE925A and LbCas12aR1138A are described in Tables 3 and 4. All reactions were carried out at 37° C. for 45 minutes, quenched with proteinase K, the samples were separated and analyzed on a 1.8% TBE Agarose gel.

TABLE 3
DNase activity of LbCas12aE925A. For targeted dsDNA cleavage: “Yes” refers to the
observation of only ~382 nucleotides and ~1318 nucleotides DNA fragments on the gel. “No”
refers to the observation of only the full length ~1700 nucleotides Zm7.1DNA and absence of
the ~382 nucleotides DNA fragment and ~1318 nucleotides DNA fragment. For ssDNA degradation:
“No” refers to the observation that M13mp18 ssDNA band intensity is comparable to the controls.

| | | | | | | Targeted | |
|---|---|---|---|---|---|---|---|
| | | | | | | dsDNA | ssDNA |
| | | | | | | cleavage | degradation |
| Assay/ | Template | Type of | | | | (N/A = Not | (N/A = Not |
| Lane | type | assay | Template | Nuclease | gRNA | applicable) | applicable) |
| 1 | dsDNA | Control | Zm7.1 | — | — | No | N/A |
| 2 | | Control | Zm7.1 | LbCas12aE925A | — | No | N/A |
| 3 | | Control | Zm7.1 | LbCas12aE925A | Cas9 gRNA | No | N/A |
| 4 | | Test | Zm7.1 | LbCas12aE925A | Cas12a gRNA | No | N/A |
| 5 | ssDNA | Control | M13mp18 | — | — | N/A | No |
| 6 | | Control | M13mp18 | LbCas12aE925A | — | N/A | No |
| 7 | | Control | M13mp18 | LbCas12aE925A | Cas9 gRNA | N/A | No |
| 8 | | Test | M13mp18 | LbCas12aE925A | Cas12a gRNA | N/A | No |
| 9 | dsDNA + | Control | Zm7.1 + | — | — | No | No |
| | ssDNA | | M13mp18 | | | | |
| 10 | | Control | Zm7.1 + | LbCas12aE925A | — | No | No |
| | | | M13mp18 | | | | |
| 11 | | Control | Zm7.1 + | LbCas12aE925A | Cas9 gRNA | No | No |
| | | | M13mp18 | | | | |
| 12 | | Test | Zm7.1 + | LbCas12aE925A | Cas12a gRNA | No | No |
| | | | M13mp18 | | | | |

TABLE 4
DNase activity of LbCas12aR1138A. Targeted dsDNA cleavage: “Yes” refers to
complete cleavage at both strands of the Zm7.1 DNA resulting in the observation of only ~382
nucleotides and ~1318 nucleotides DNA fragments on the gel. “No” refers to the observation of
only the full length ~1700 nucleotides Zm7.1DNA. ‘Partial’ refers to the observation
of ~1700 nucleotides full length Zm7.1 DNA, ~382 nucleotides DNA fragment and ~1318
nucleotides DNA fragment. For ssDNase activity, “No” refers to observation
that M13mp18 ssDNA band intensity is comparable to that seen in the controls.
Results represent assays where nuclease: DNA ratio was 1:60 and 1:100.

| | | | | | | Targeted | |
|---|---|---|---|---|---|---|---|
| | | | | | | dsDNA | |
| | | | | | | cleavage | ssDNase |
| | | | | | | activity | activity |
| Assay/ | Template | Type of | | | | (N/A = Not | (N/A = Not |
| Lane | type | assay | Template | Nuclease | gRNA | applicable) | applicable) |
| 1 | dsDNA | Control | Zm7.1 | — | — | No | N/A |
| 2 | | Control | Zm7.1 | LbCas12aR1138A | | No | N/A |
| 3 | | Control | Zm7.1 | LbCas12aR1138A | Cas9 gRNA | No | N/A |
| 4 | | Test | Zm7.1 | LbCas12aR1138A | Cas12a gRNA | Partial | N/A |
| 5 | ssDNA | Control | M13mp18 | — | — | N/A | No |
| 6 | | Control | M13mp18 | LbCas12aR1138A | | N/A | No |
| 7 | | Control | M13mp18 | LbCas12aR1138A | Cas9 gRNA | N/A | No |
| 8 | | Test | M13mp18 | LbCas12aR1138A | Cas12a gRNA | N/A | No |
| 9 | dsDNA + | Control | Zm7.1 + | — | — | No | No |
| | ssDNA | | M13mp18 | | | | |
| 10 | | Control | Zm7.1 + | LbCas12aR1138A | — | No | No |
| | | | M13mp18 | | | | |
| 11 | | Control | Zm7.1 + | LbCas12aR1138A | Cas9 gRNA | No | No |
| | | | M13mp18 | | | | |
| 12 | | Test | Zm7.1 + | LbCas12aR1138A | Cas12a gRNA | Partial | No |
| | | | M13mp18 | | | | |

As shown in Table 3 (Assays 4 and 12), in reactions comprising Zm7.1 template DNA with LbCas12aE925A and its cognate gRNA, only the full length ˜1700 nucleotides Zm7.1 DNA was observed. This data suggests that at 60:1 protein to DNA ratio and in the presence of its cognate gRNA, LbCas12aE925A did not cleave both strands of the target dsDNA. As shown in Table 3 (Assays 8 and 12), in reactions comprising the M13mp18 ssDNA with LbCas12aE925A and its gRNA, the full length M13mp18 ssDNA band intensity was comparable to that observed in the controls. This suggests that at 60:1 protein to DNA ratio, LbCas12aE925A did not degrade the M13mp18ssDNA in the presence of the cognate Cas12a guide RNA.

As described in Table 4 (Assays 4 and 12), in reactions comprising Zm7.1 template DNA, LbCas12aR1138A and its cognate gRNA, three bands were observed: the full length 1700 nucleotides Zm7.1 DNA, an ˜383 nucleotides band and an ˜1318 nucleotides band. This data suggests that despite being predicted as a nickase, LbCas12aR1138A still possessed dsDNA cleavage activity resulting in the site directed cleavage of both strands of the Zm7.1 dsDNA. The dsDNA processing activity of LbCas12aR1138A appears to be less than wtLbCas12a as evidenced by the presence of some amount of uncut Zm7.1 dsDNA. As described in Table 4 (Assays 8 and 12), in reactions comprising the M13mp18ssDNA with LbCas12aR1138A and its gRNA, the intensity of the ssDNA band was comparable to that seen in the controls. This suggests that substitution of alanine for arginine at position 1138 led to significant loss in the ssDNase activity of LbCas12a. These results were consistent when the protein to DNA ratio was increased from 60:1 to 100:1 (see Table 4).

### Example 4. Effect of Time and Temperature on LbCas12a and LbCas12aR1138A dsDNA Processing and ssDNase Activity

Temperature is known to modulate the activity of Cas12a (see, for example, Moreno-Mateos et. al. 2017, DOI: 10.1038/s41467-017-01836-2). To compare the DNase activity of LbCas12a and LbCas12aR1138A, time-course assays was carried out with the two proteins and the processing activity was assayed at 25° C. and 37° C. Each test reaction mixture comprised the purified LbCas12a protein or LbCas12aR1138A variant mixed with Zm7.1 dsDNA or M13mp18 ssDNA at a ratio of 60:1 along with Cas12a-zm7.1 gRNA. Three controls were run in parallel. The first control lacked the Cas12a nuclease and gRNA, the second control comprised the template and nuclease but lacked the cognate Cas12a gRNA and the third control included the nuclease and template with a Cas9 guide RNA that is not known in the literature to be recognized by Cas12a. The test and control reaction mixtures were incubated at either 25° C. or 37° C. and quenched with proteinase K after 10 minutes, 20 minutes, 40 minutes, 90 minutes or 180 minutes. The samples were separated and visually analyzed on a 1.8% TBE Agarose gel. The test assay results are described in Table 5.

TABLE 5
Time course assays comparing DNase activity of LbCas12a and LbCas12a-R1138A at
25° C. and 37° C. in the presence of the cognate Cas12a-gRNA. For reactions comprising the dsDNA
template: “−” refers to observation of only the full length ~1700 nucleotides Zm7.1DNA; “+”,
“++”, “+++” represent partial dsDNA processing and observation of ~1700, ~382 and ~1318
nucleotides bands, where “+” refers to weak processing, “++” refers to moderate processing,
“+++” refers to strong processing, and “+++✓” refers to complete processing and observation of
only ~382 nucleotides and ~1318 nucleotides DNA fragments on the gel. For reactions comprising
the ssDNA: “−” refers to observation that M13mp18 ssDNA band intensity is comparable to the
controls; “+++” refers to partial processing and observation that ssDNA band intensity is less than
that observed in the controls; and ‘+++✓″ refers to no ssDNA observed.

| | | Time | | | | | | | | | |
|---|---|---|---|---|---|---|---|---|---|---|---|
| | Temperature | 10 mins | | 20 mins | | 40 mins | | 90 mins | | 180 mins | |
| | ↓ | Zm7.1 | M13 | Zm7.1 | | Zm7.1 | | Zm7.1 | | Zm7.1 | |
| Assay | Template | (dsDNA) | (ssDNA) | (dsDNA) | M13mp18 | (dsDNA) | M13mp18 | (dsDNA) | M13mp18 | (dsDNA) | M13mp18 |
| LbCas12a + | 25° C. | ++ | +++ | ++ | +++✓ | +++ | +++✓ | +++ | +++✓ | +++✓ | +++✓ |
| Cas12a-gRNA | 37° C. | +++ | +++ | +++ | +++✓ | +++✓ | +++✓ | +++✓ | +++✓ | +++✓ | +++✓ |
| LbCas12a | 25° C. | − | − | − | − | + | − | ++ | − | +++ | − |
| R1138A + | | | | | | | | | | | |
| Cas 12a-gRNA | 37° C. | + | ~ | ++ | − | ++ | − | +++ | − | +++✓ | − |

As shown in Table 5, for LbCas12a, the targeted dsDNA processing of Zm7.1 reached completion at 40 mins when the reactions were incubated at 37° C. For reactions incubated at 25° C., complete processing was achieved by 180 minutes suggesting a modest decrease in activity at 25° C. The ssDNase activity of LbCas12a was comparable across the two temperatures tested and reached completion by 20 minutes.

The targeted dsDNA processing activity of LbCas12aR1138A was slower than the wildtype at both temperatures though at 37° C. it reached completion by 180 minutes. No evidence of ssDNase activity was noted for the LbCas12aR1138A variant at all tested time points and temperatures. For all the control assays, no ssDNase or targeted dsDNase activity was observed at all tested time points and temperatures.

### Example 5. Testing DNase Activity of Additional LbCas12a Variants

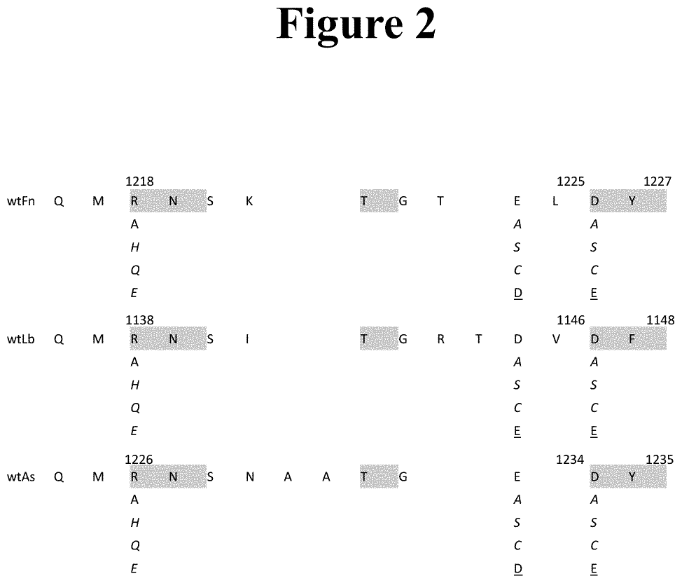

Analysis of the crystal structure of FnCas12a and point mutations has revealed that the DNA nuclease activity takes place in a pocket at the interface between the RuvC and Nuc domains (see Stella et al., Nature, 546:559-563 (2017)). The R1138 residue in LbCas12a resides within this interface. A series of substitutions were designed at the R1138 position so as to alter the charge, change donor capacity and change potential catalytic residue length with the goal of altering ssDNase activity (FIG. 2). Additional mutants were also created at residue D1146 and D1148, both of which reside within the RuvC-Nuc interface. All mutants were investigated for their dsDNase and ssDNase activity by the in-vitro DNase assay described in Experiment 1. Each test reaction mixture comprised the purified LbCas12a variant mixed with Zm7.1 dsDNA or M13mp18ssDNA at a ratio of 60:1 along with Cas12a-zm7.1 gRNA. Three negative controls were run in parallel. The first control comprised only the template and lacked the Cas12a nuclease and gRNA, the second control comprised the template and nuclease but lacked the cognate Cas12a gRNA and the third control included the nuclease and template with a Cas9 guide RNA that is not known in the literature to be recognized by Cas12a. The test and control reaction mixtures were incubated at 37° C. and quenched with proteinase K after 45 minutes. The samples were separated and visually analyzed on a 1.8% TBE Agarose gel. The variants tested and results are disclosed in Table 6.

TABLE 6
DNase activity of LbCas12a variants in the presence of the cognate guide RNA. For
targeted dsDNA cleavage: “Yes” refers to the observation of only ~382 bp and ~1318 bp
DNA fragments on the gel. “No” refers to the observation of only the full length ~1700 bp
Zm7.1DNA. ‘Partial’ refers to the observation of ~1700 bp full length Zm7.1
DNA, ~382 bp DNA fragment and ~1318 bp DNA fragment. For ssDNase activity, “Yes”
refers to the observation that M13mp18 ssDNA band was either absent or its
intensity was significantly less than that observed in the controls; “No” refers to
the observation that M13mp18 ssDNA band intensity is comparable to the controls.

| | | Targeted dsDNA | |
|---|---|---|---|
| | | cleavage activity | ssDNase activity |
| Nuclease + | | (N/A = not | (N/A = not |
| Cas12a-gRNA | Template | applicable) | applicable) |
| LbCas12a | Zm7.1 (dsDNA) | Yes | N/A |
| (SEQ ID NO: 2) | M13mp18 (ssDNA) | N/A | Yes |
| LbCas12a-R1138A | Zm7.1 (dsDNA) | Partial | — |
| (SEQ ID NO: 8) | M13mp18 (ssDNA) | N/A | No |
| LbCas12a-R1138H | Zm7.1 (dsDNA) | Partial | N/A |
| (SEQ ID NO: 10) | M13mp18 (ssDNA) | N/A | No |
| LbCas12a-R1138Q | Zm7.1 (dsDNA) | No | N/A |
| (SEQ ID NO: 11) | M13mp18 (ssDNA) | N/A | No |
| LbCas12a-R1138E | Zm7.1 (dsDNA) | Partial | N/A |
| (SEQ ID NO: 12) | M13mp18 (ssDNA) | N/A | No |
| LbCas12a-D1146A | Zm7.1 (dsDNA) | Yes | N/A |
| (SEQ ID NO: 13) | M13mp18 (ssDNA) | N/A | Yes |
| LbCas12a-D1146S | Zm7.1 (dsDNA) | Yes | N/A |
| (SEQ ID NO: 14) | M13mp18 (ssDNA) | N/A | Yes |
| LbCas12a-D1146C | Zm7.1 (dsDNA) | Yes | N/A |
| (SEQ ID NO: 15) | M13mp18 (ssDNA) | N/A | Yes |
| LbCas12a-D1146E | Zm7.1 (dsDNA) | Yes | N/A |
| (SEQ ID NO: 16) | M13mp18 (ssDNA) | N/A | Yes |
| LbCas12a-D1148A | Zm7.1 (dsDNA) | Yes | N/A |
| (SEQ ID NO: 17) | M13mp18 (ssDNA) | N/A | Yes |
| LbCas12a-D1148S | Zm7.1 (dsDNA) | Yes | N/A |
| (SEQ ID NO: 18) | M13mp18 (ssDNA) | N/A | Yes |
| LbCas12a-D1148C | Zm7.1 (dsDNA) | Yes | N/A |
| (SEQ ID NO: 19) | M13mp18 (ssDNA) | N/A | Yes |
| LbCas12a-D1148E | Zm7.1 (dsDNA) | Yes | N/A |
| (SEQ ID NO: 20) | M13mp18 (ssDNA) | N/A | Yes |

Among the protein variants tested, LbCas12a-R1138A and LbCas12a-R1138H maintained dsDNA cutting activity while ssDNase activity was not observed (Table 6). Time course assays were carried out with LbCas12a-R1138H, as described in Example 4 and it was noted that the targeted dsDNA processing of LbCpf-1R1138H reached completion by 180 minutes. ssDNase activity was not observed at 180 minutes.

### Example 6. DNase Activity of LbCas12a and LbCas12a Variants in the Presence of Different Guide RNAs and Cognate Target Sites

The experiments described in Examples 1-5 test the DNase activity of Cas12a in the presence of the Cas12a-Zm7.1 gRNA. To investigate if this activity was independent of guide RNA sequence, the in vitro cutting activity of LbCas12a, LbCas12aR1138A and LbCas12aR1138H was tested in the presence of six additional individual gRNAs. Three synthetic dsDNA substrates were created for this purpose. E_1088 was a 1716 nucleotide PCR product that comprised 3 unique target sites: ZmTS1; ZmTS2 site and ZmTS3 site. Each TS site was originally identified in the corn genome, was 23 nucleotides long, and comprised a Cas12a PAM sequence TTTN 5′ to the target sequence. gRNAs were designed to recognize each target site and these are described in Table 7. Complete targeted cleavage of E_1088 by Cas12a and each gRNA would result in digestions products 1 and 2 described in Table 7.

TABLE 7
E_1088 dsDNA with its target sites and expected digestion products
following complete processing by Cas12a-guide RNA complex.

| | Position of | | E_1088 Digestion | |
|---|---|---|---|---|
| | target site | | product size | |
| | on E_1088 | | Product 1 | Product 2 |
| Target site | (nucleotides) | Cognate gRNA | (nucleotides) | (nucleotides) |
| ZmTS1 | 507 | Cas12a ZmTS2 gRNA | 1209 | 507 |
| ZmTS2 | 611 | Cas12a ZmTS3 gRNA | 1105 | 611 |
| ZmTS3 | 717 | Cas12a ZmTS4 gRNA | 999 | 717 |

E_1090 was a 1702 nucleotides PCR product that comprised 3 unique target sites: GmTS1; GmTS2 site and GmTS3 site. The 23 nucleotides long GmTS1, 2 and 3 sites were originally identified in the soy genome and each comprised a Cas12a PAM sequence TTTN, 5′ to the target site. gRNAs were designed to recognize each target site and are described in Table 8. Complete targeted-cleavage of E_1090 dsDNA by LbCas12a and each gRNA would result in digestions products described in Table 8.

TABLE 8
E_l090 dsDNA with target sites and expected digestion products
following complete processing by Cas12a-guide RNA complex.

| | | | E_1090 Digestion | |
|---|---|---|---|---|
| | Position of | | product size | |
| | target site | | Product 1 | Product 2 |
| Target | on E_1090 | | (nucleo- | (nucleo- |
| site | (nucleotides) | Cognate gRNA | tides) | tides) |
| GmTS1 | 409 | Cas12a ZmTS2 gRNA | 1293 | 409 |
| GmTS2 | 539 | Cas12a ZmTS3 gRNA | 1163 | 539 |
| GmTS3 | 650 | Cas12a ZmTS4 gRNA | 1052 | 650 |

E_1089 was a 1747 nucleotides PCR product that comprised 7 unique target sites: Cas12a-Zm7.1; GmTS1; ZmTS1; GmTS2 site, ZmTS2, ZmTS3 and GmTS3 site. Complete targeted-cleavage of E_1090 dsDNA by LbCas12a and each gRNA would result in digestions products described in Table 9.

TABLE 9
E_1090 dsDNA with target sites and expected digestion products
following complete processing by Cas12a-guide RNA complex.

| | | | E_1090 Digestion | |
|---|---|---|---|---|
| | Position of | | product size | |
| | target site | | Product 1 | Product 2 |
| Target | on E_1090 | | (nucleo- | (nucleo- |
| site | (nucleotides) | Cognate gRNA | tides) | tides) |
| Cas12a- | 380 | Cas12a Zm7.1 gRNA | 1367 | 380 |
| Zm7.1 | | | | |
| GmTS1 | 409 | Cas12a gmTS1 gRNA | 1338 | 409 |
| ZmTS1 | 511 | Cas12a ZmTS1 gRNA | 1236 | 511 |
| GmTS2 | 566 | Cas12a GmTS2 gRNA | 1181 | 566 |
| ZmTS2 | 619 | Cas12a ZmTS2 gRNA | 1128 | 619 |
| ZmTS3 | 684 | Cas12a ZmTS3 gRNA | 1063 | 684 |
| GmTS3 | 762 | Cas12a GmTS3 gRNA | 985 | 762 |

The in vitro DNase assay described in Example 1 was used to investigate the guide RNA directed DNA cutting activity of LbCas12a, LbCas12a R1138A and LbCas12aR1138H on the three dsDNA templates described above as well as M13 ssDNA. The experimental set up is described in Table 10. For test assays, purified LbCas12a protein or LbCas12a variants were mixed with gRNAs and either of the three dsDNA templates or M13mp18ssDNA template. The protein to DNA ratio was maintained at 60:1. Two control reactions were run in parallel for each test assay. The first control lacked both the nuclease and gRNA while the second control lacked the gRNA. All reactions were incubated at 37° C. for 45 minutes and quenched with proteinase K. The samples were separated and analyzed on a 1.8% TBE Agarose gel. The results are described in Table 10.

TABLE 10
Targeted ds DNA cleavage and ssDNase activity of LbCas12a, LbCas12aR1138A and
LbCas12aR1138H in the presence of different gRNAs. Table legend: For dsDNA cleavage: “No”
refers to the observation of a band corresponding to the full length template DNA. “Yes.
Complete” refers to the observation of bands corresponding to the cleavage products 1 and 2. “Yes.
Partial” refers to the observation of bands corresponding to full length template and cleavage
products 1 and 2. For ssDNase activity, “No” refers to observation that ssDNA band intensity was
comparable to the controls; “Yes” refers to the observation that ssDNA band was absent or the
band intensity less than that observed in the controls.

| | | | | Targeted dsDNA | ssDNA |
|---|---|---|---|---|---|
| | | | | cleavage | degradation |
| | Type of | | | (N/A = Not | (N/A = Not |
| Template | assay | Nuclease | gRNA | applicable) | applicable) |
| E_1088 | Control | — | — | No | |
| (dsDNA) | Control | LbCas12a | — | No | |
| | Test | LbCas12a | ZmTS1 | Yes. Complete | N/A |
| | | | gRNA | | |
| | Test | LbCas12a | ZmTS2 | Yes. Complete | N/A |
| | | | gRNA | | |
| | Test | LbCas12a | ZmTS3 | Yes. Complete | N/A |
| | | | gRNA | | |
| M13mp18 | Control | — | — | N/A | No |
| (ssDNA) | Control | LbCas12a | — | N/A | No |
| | Test | LbCas12a | ZmTS1 | N/A | Yes |
| | | | gRNA | | |
| | Test | LbCas12a | ZmTS2 gRNA | N/A | Yes |
| | Test | LbCas12a | ZmTS3 | N/A | Yes |
| | | | gRNA | | |
| E_1088 | Control | — | — | No | N/A |
| (dsDNA) | Control | LbCas12aR1138A | — | No | N/A |
| | Test | LbCas12aR1138A | ZmTS1 | Yes. Partial | N/A |
| | | | gRNA | | |
| | Test | LbCas12aR1138A | ZmTS2 | Yes. Partial | N/A |
| | | | gRNA | | |
| | Test | LbCas12aR1138A | ZmTS3 | Yes. Partial | N/A |
| | | | gRNA | | |
| M13mp18 | Control | — | — | No | No |
| (ssDNA) | | | | | |
| | Control | LbCas12aR1138A | — | No | No |
| | Test | LbCas12aR1138A | ZmTS1 | N/A | No |
| | | | gRNA | | |
| | Test | LbCas12aR1138A | ZmTS2 | N/A | No |
| | | | gRNA | | |
| | Test | LbCas12aR1138A | ZmTS3 | N/A | No |
| | | | gRNA | | |
| E_1088 | Control | — | — | No | N/A |
| (dsDNA) | Control | LbCas12aR1138H | — | No | N/A |
| | Test | LbCas12aR1138H | ZmTS1 | Yes. Partial | N/A |
| | | | gRNA | | |
| | Test | LbCas12aR1138H | ZmTS2 | Yes. Partial | N/A |
| | | | gRNA | | |
| | Test | LbCas12aR1138H | ZmTS3 | Yes. Partial | N/A |
| | | | gRNA | | |
| M13mp18 | Control | — | — | N/A | No |
| (ssDNA) | Control | LbCas12aR1138H | — | N/A | No |
| | Test | LbCas12aR1138H | ZmTS1 | N/A | No |
| | | | gRNA | | |
| | Test | LbCas12aR1138H | ZmTS2 | N/A | No |
| | | | gRNA | | |
| | Test | LbCas12aR1138H | ZmTS3 | N/A | No |
| | | | gRNA | | |
| E_1090 | Control | — | — | No | N/A |
| (dsDNA) | Control | LbCas12a | — | No | N/A |
| | Test | LbCas12a | GmTS1 | Yes. Complete | N/A |
| | | | gRNA | | |
| | Test | LbCas12a | GmTS2 | Yes. Complete | N/A |
| | | | gRNA | | |
| | Test | LbCas12a | GmTS3 | Yes. Complete | N/A |
| | | | gRNA | | |
| M13mp18 | Control | — | — | N/A | No |
| (ssDNA) | Control | LbCas12a | — | N/A | No |
| | Test | LbCas12a | GmTS1 | N/A | Yes |
| | | | gRNA | | |
| | Test | LbCas12a | GmTS2 | N/A | Yes |
| | | | gRNA | | |
| | Test | LbCas12a | GmTS3 | N/A | Yes |
| | | | gRNA | | |
| E_1090 | Control | — | — | No | N/A |
| (dsDNA) | Control | LbCas12aR1138A | — | No | N/A |
| | Test | LbCas12aR1138A | GmTS1 | Yes. Partial | N/A |
| | | | gRNA | | |
| | Test | LbCas12aR1138A | GmTS2 | Yes. Partial | N/A |
| | | | gRNA | | |
| | Test | LbCas12aR1138A | GmTS3 | Yes. Partial | N/A |
| | | | gRNA | | |
| M13mp18 | Control | — | — | N/A | No |
| (ssDNA) | Control | LbCas12aR1138A | — | N/A | No |
| | Test | LbCas12aR1138A | GmTS1 | N/A | No |
| | | | gRNA | | |
| | Test | LbCas12aR1138A | GmTS2 | N/A | No |
| | | | gRNA | | |
| | Test | LbCas12aR1138A | GmTS3 | N/A | No |
| | | | gRNA | | |
| E_1090 | Control | — | — | No | N/A |
| (dsDNA) | Control | LbCas12aR1138H | — | No | N/A |
| | Test | LbCas12aR1138H | GmTS1 | Yes. Partial | N/A |
| | | | gRNA | | |
| | Test | LbCas12aR1138H | GmTS2 | Yes. Partial | N/A |
| | | | gRNA | | |
| | Test | LbCas12aR1138H | GmTS3 | Yes. Partial | N/A |
| | | | gRNA | | |
| M13mp18 | Control | — | — | N/A | No |
| (ssDNA) | Control | LbCas12aR1138H | — | N/A | No |
| | Test | LbCas12aR1138H | GmTS1 | N/A | No |
| | | | gRNA | | |
| | Test | LbCas12aR1138H | GmTS2 | N/A | No |
| | | | gRNA | | |
| | Test | LbCas12aR1138H | GmTS3 | N/A | No |
| | | | gRNA | | |
| E_1089 | Control | — | — | No | N/A |
| (dsDNA) | Control | LbCas12a | — | No | N/A |
| | Test | LbCas12a | ZmTS1 | Yes. Complete | N/A |
| | | | gRNA | | |
| | Test | LbCas12a | ZmTS2 | Yes. Complete | N/A |
| | | | gRNA | | |
| | Test | LbCas12a | ZmTS3 | Yes. Complete | N/A |
| | | | gRNA | | |
| | Test | LbCas12a | GmTS1 | Yes. Complete | N/A |
| | | | gRNA | | |
| | Test | LbCas12a | GmTS2 | Yes. Complete | N/A |
| | | | gRNA | | |
| | Test | LbCas12a | GmTS3 | Yes. Complete | N/A |
| | | | gRNA | | |
| E_1089 | Control | — | — | N/A | N/A |
| (dsDNA) | Control | LbCas12aR1138A | — | N/A | N/A |
| | Test | LbCas12aR1138A | ZmTS1 | Yes. Partial | N/A |
| | | | gRNA | | |
| | Test | LbCas12aR1138A | ZmTS2 | Yes. Partial | N/A |
| | | | gRNA | | |
| | Test | LbCas12aR1138A | ZmTS3 | Yes. Partial | N/A |
| | | | gRNA | | |
| | Test | LbCas12aR1138A | GmTS1 | Yes. Partial | N/A |
| | | | gRNA | | |
| | Test | LbCas12aR1138A | GmTS2 | Yes. Partial | N/A |
| | | | gRNA | | |
| | Test | LbCas12aR1138A | GmTS3 | Yes. Partial | N/A |
| | | | gRNA | | |
| E_1089 | Control | — | — | No | N/A |
| (dsDNA) | Control | LbCas12aR1138H | — | No | N/A |
| | Test | LbCas12aR1138H | ZmTS1 | Yes. Partial | N/A |
| | | | gRNA | | |
| | Test | LbCas12aR1138H | ZmTS2 | Yes. Partial | N/A |
| | | | gRNA | | |
| | Test | LbCas12aR1138H | ZmTS3 | Yes. Partial | N/A |
| | | | gRNA | | |
| | Test | LbCas12aR1138H | GmTS1 | Yes. Partial | N/A |
| | | | gRNA | | |
| | Test | LbCas12aR1138H | GmTS2 | Yes. Partial | N/A |
| | | | gRNA | | |
| | Test | LbCas12aR1138H | GmTS3 | Yes. Partial | N/A |
| | | | gRNA | | |

The results from the in vitro DNase assays show that, when LbCas12a is complexed with any of the six tested gRNAs, it completes targeted dsDNA cleavage of all tested dsDNA templates within 45 minutes and degrades ssDNA. Under the same conditions, LbCas12aR1138A and LbCas12aR1138H show partial cleavage of dsDNA and there was no visual evidence of ssDNA degradation.

### Example 7. LbCas12a, LbCas12aR1138A, and LbCas12aR1138H Cleavage Activity in Soy Protoplasts

To test whether LbCas12aR1138A and LbCas12aR1138H could recognize, cleave, and mutate soy chromosomal DNA in the presence of gRNAs, three different genomic target sites were targeted for cleavage and examined by deep sequencing for the presence of mutations indicative of cleavage. Wildtype LbCas12a was used as a positive control and dead LbCas12a(dLbCas12a) was used as a negative control. LbCas12a shows a preference for the TTTN PAM sequence therefore, target sites GmTS1, GmTS2 and GmTS3 were chosen based on the occurrence of the appropriate PAM sequence at the 5′ end. GmTS1 gRNA, GmTS2 gRNA, GmTS3 gRNAs were designed to target the three sites.

TABLE 11
Assays to evaluate the editing efficiency of LbCas12a,
LbCas12aR1138A and LbCas12aR1138H in soy protoplasts.
“—” = noguide RNA (gRNA) control.

| Nuclease | gRNA | Target site | Replicates |
|---|---|---|---|
| dLbCas12a | — | — | 18 |
| | GmTS1 gRNA | GmTS1 | 6 |
| | GmTS2 gRNA | GmTS2 | 6 |
| | GmTS3 gRNA | GmTS3 | 6 |
| LbCas12a | — | — | 17 |
| | GmTS1 gRNA | GmTS1 | 6 |
| | GmTS2 gRNA | GmTS2 | 6 |
| | GmTS3 gRNA | GmTS3 | 6 |
| LbCas12aR1138A | — | — | 18 |
| | GmTS1 gRNA | GmTS1 | 6 |
| | GmTS2 gRNA | GmTS2 | 6 |
| | GmTS3 gRNA | GmTS3 | 6 |
| LbCas12aR1138H | — | — | 18 |
| | GmTS1 gRNA | GmTS1 | 6 |
| | GmTS2 gRNA | GmTS2 | 5 |
| | GmTS3 gRNA | GmTS3 | 6 |

The LbCas12a variants described in Table 11 were expressed and purified from E. coli. The nucleases were mixed with the corresponding gRNAs at a 1:2 (gRNA:nuclease) ratio to form Ribonucleoprotein complexes and transformed into soy protoplasts using standard polyethylene glycol (PEG) mediated transformation. For quantifying transformation frequency, a vector containing a GFP expression cassette was co-delivered. As controls, protoplasts were transformed with just the nucleases and no guide RNA. Multiple technical replicates were carried out for each assay. Following transformation, the protoplasts were incubated in the dark in incubation buffer and harvested after 48 hours. Genomic DNA was isolated and the region surrounding the intended target site was amplified and deep sequenced by Illumina sequencing using standard methods known in the art. The resulting reads were assessed for the presence of mutations, specifically insertions or deletions (INDELs) at the expected site of cleavage. Table 12 and FIG. 3 summarize the mean INDEL rates observed for each test treatment with nuclease and guide RNA at each site.

TABLE 12
Mean INDEL rate with standard deviation in parentheses.

| Nuclease | GmTS1 | GmTS2 | GmTS3 |
|---|---|---|---|
| dLbCas12a | 0.085(0.009) | 0.058(0.013) | 0.038(0.012) |
| LbCas12a | 7.397(1.248) | 7.066(2.795) | 24.526(1.056) |
| LbCas12aR1138A | 2.101(0.811) | 0.476(0.398) | 3.315(0.589) |
| LbCas12aR1138H | 7.437(1.42) | 1.516(0.681) | 7.444(0.744) |

To assess the statistical significance of the difference between treatment and no-gRNA control INDEL rates for the R1138 variants, Wilcoxon Rank Sum tests were performed for each variant within each site. Both un-adjusted and Holms-Bonferroni adjusted p-values are provided. Results are summarized in Table 13 and indicate that the R1138A and R1138H variants have significantly higher INDEL rates compared to their respective no-gRNA controls.

TABLE 13
LbCas12aR1138 variant vs. no-gRNA control. Two-sided Wilcoxon
Rank sum test with Holm adjustment.

| Target site | Nuclease | Statistic | P-value | P adjust |
|---|---|---|---|---|
| GmTS1 | LbCas12aR1138A | 36 | 0.0022 | 0.0130 |
| GmTS1 | LbCas12aR1138H | 36 | 0.0022 | 0.0130 |
| GmTS2 | LbCas12aR1138A | 36 | 0.0022 | 0.0130 |
| GmTS2 | LbCas12aR1138H | 30 | 0.0043 | 0.0130 |
| GmTS3 | LbCas12aR1138A | 36 | 0.0022 | 0.0130 |
| GmTS3 | LbCas12aR1138H | 36 | 0.0022 | 0.0130 |

Within each target site, differences in INDEL rates of the two R1138 variants were assessed using the Wilcoxon Rank Sum test (Table 14). INDEL rates for R1137H was significantly higher that R1138A across all sites.

TABLE 14
Two sided Wilcoxon Rank Sum: LbCas12aR1138A vs LbCas12aR1138H

| Target site | Statistic | P-value | P adjust |
|---|---|---|---|
| GmTS1 | 0 | 0.0022 | 0.0065 |
| GmTS2 | 2 | 0.0173 | 0.0173 |
| GmTS3 | 0 | 0.0022 | 0.0065 |

### Example 8: Role of Magnesium in Non-Target ssDNA Cleavage

The role of magnesium in non-target ssDNA cleavage by wildtype RNA-guided CRISPR nucleases was examined. The activity of wildtype RNA-guided CRISPR nucleases SpCas9 (SEQ ID NO:22) and LbCas12a (SEQ ID NO: 2) were investigated using the in vitro assays described in Example 1 and 2. The open reading frame of SpCas9 sequence was codon-optimized for optimal expression in E. coli cells (SEQ ID NO: 23). A histidine tag sequence (SEQ ID NO: 4) was introduced at the 5′ end of the gene. Additionally, two nuclear localization signals (NLS) (SEQ ID NOs: 5 and 6) were introduced at the 5′ and 3′ ends of SpCas9 open reading frame resulting in HIStag:NLS:SpCas9:NLS. The design of HIStag:NLS:LbCas12a:NLS fusion protein has been described in Example 1. E. coli purified LbCas12a or SpCas9 fusion proteins were subsequently assembled with or without the nuclease-appropriate gRNA (100 μM) and incubated with the dsDNA (26.7 nM), M13 ssDNA (12.54 nM), or a combination of dsDNA and ssDNA in cleavage buffer comprising 20 mM HEPES and 0.5 mM DTT and either 0.02 mM MgCl₂ or 10 mM MgCl₂. The protein amounts were adjusted to accommodate the specific protein to DNA ratio of 60:1 for each reaction. The reactions were carried out at 37° C. for 45 minutes and quenched with proteinase K treatment at 65° C. for 15 minutes. The samples were separated and analyzed on a 1.8% TBE Agarose gel. The observations for LbCas12a are summarized in Table 15 and the observations for SpCas9 are summarized in Table 16.

In the presence of 0.02 mM MgCl₂ non-specific ssDNA cleavage was not observed for SpCas9 or LbCas12a. In contrast, non-specific ssDNA cleavage was observed for both SpCas9 and LbCas12a in the presence of 10 mM MgCl₂ when paired with the nuclease-appropriate gRNA.

TABLE 15
Role of Magnesium on the DNase activity assay of LbCas12a. For targeted dsDNA
cleavage, “Yes” refers to the observation of only the ~382 bp and ~1318 bp DNA fragments on
the gel. “No” refers to the observation of only the full length ~1700 bpZm7.1DNA. For ssDNase
activity, “Yes” refers to the observation that M13mp18 ssDNA band was either absent or its
intensity was less than that observed in the controls. “No” refers to observation that M13mp18
ssDNA band intensity was comparable to the intensity observed in the controls.

| | | | | | | | Targeted | |
|---|---|---|---|---|---|---|---|---|
| | | | | | | | dsDNA | |
| | | | | | | | cleavage | ssDNase |
| | | | | | | | activity | activity |
| Assay/ | Template | Type of | | | | | (N/A = Not | (N/A = Not |
| Lane | type | assay | Template | Nuclease | gRNA | MgCl₂ | applicable) | applicable) |
| 1 | dsDNA | Control | Zm7.1 | — | — | 10 mM | No | N/A |
| 2 | | Control | Zm7.1 | LbCas12a | — | 10 mM | No | N/A |
| 3 | | Control | Zm7.1 | LbCas12a | Cas9 gRNA | 10 mM | No | N/A |
| 4 | | Test | Zm7.1 | LbCas12a | Cas12a gRNA | 10 mM | Yes | N/A |
| 5 | dsDNA | Control | Zm7.1 | — | — | 0.02 mM | No | N/A |
| 6 | | Control | Zm7.1 | LbCas12a | — | | No | N/A |
| 7 | | Control | Zm7.1 | LbCas12a | Cas9 gRNA | | No | N/A |
| 8 | | Test | Zm7.1 | LbCas12a | Cas12a gRNA | | Yes | N/A |
| 9 | ssDNA | Control | M13mp18 | — | — | 10 mM | N/A | No |
| 10 | | Control | M13mp18 | LbCas12a | — | | N/A | No |
| 11 | | Control | M13mp18 | LbCas12a | Cas9 gRNA | | N/A | No |
| 12 | | Test | M13mp18 | LbCas12a | Cas12a gRNA | | N/A | Yes |
| 13 | ssDNA | Control | M13mp18 | — | — | 0.02 mM | N/A | No |
| 14 | | Control | M13mp18 | LbCas12a | — | | N/A | No |
| 15 | | Control | M13mp18 | LbCas12a | Cas9 gRNA | | N/A | No |
| 16 | | Test | M13mp18 | LbCas12a | Cas12a gRNA | | N/A | No |
| 17 | dsDNA + | Control | Zm7.1 + | — | — | 10 mM | No | No |
| | ssDNA | | M13mp18 | | | MgCl2 | | |
| 18 | | Control | Zm7.1 + | LbCas12a | — | | No | No |
| | | | M13mp18 | | | | | |
| 19 | | Control | Zm7.1 + | LbCas12a | Cas9 gRNA | | No | No |
| | | | M13mp18 | | | | | |
| 20 | | Test | Zm7.1 + | LbCas12a | Cas12a gRNA | | Yes | Yes |
| | | | M13mp18 | | | | | |
| 21 | dsDNA + | Control | Zm7.1 + | — | — | 0.2 mM | No | No |
| | ssDNA | | M13mp18 | | | MgCl2 | | |
| 22 | | Control | Zm7.1 + | LbCas12a | — | | No | No |
| | | | M13mp18 | | | | | |
| 23 | | Control | Zm7.1 + | LbCas12a | Cas9 gRNA | | No | No |
| | | | M13mp18 | | | | | |
| 24 | | Test | Zm7.1 + | LbCas12a | Cas12a gRNA | | Yes | No |
| | | | M13mp18 | | | | | |

TABLE 16
Role of magnesium on the DNase activity assay of SpCas9. For targeted dsDNA
cleavage, “Yes” refers to the observation of only the ~350 bp and ~1350 bp DNA fragments on
the gel. “Yes. Partial” refers to the observation of ~1700bp, ~350 bp and ~1350 bp DNA fragments.
“No” refers to the observation of the full length ~1700 bpZm7.1DNA and absence of the ~350 bp
DNA fragment and ~1350 bp DNA fragment. For ssDNase activity, “Yes” refers to the observation
that M13mp18 ssDNA band was either absent or its intensity was less than that observed in the
controls. “No” refers to observation that M13mp18 ssDNA band intensity was comparable to the
intensity observed in the controls.

| | | | | | | | Targeted | |
|---|---|---|---|---|---|---|---|---|
| | | | | | | | dsDNA | |
| | | | | | | | cleavage | ssDNase |
| | | | | | | | activity | activity |
| Assay/ | Template | Type of | | | | | (N/A = Not | (N/A = Not |
| Lane | type | assay | Template | Nuclease | gRNA | MgCl₂ | applicable) | applicable) |
| 1 | dsDNA | Control | Zm7.1 | — | — | 10 mM | No | N/A |
| 2 | | Control | | SpCas9 | — | | No | N/A |
| 3 | | Test | | SpCas9 | Cas9 gRNA | | Yes | N/A |
| 4 | | Control | | SpCas9 | Cas12a gRNA | | No | N/A |
| 5 | dsDNA | Control | Zm7.1 | — | — | 0.02 mM | No | N/A |
| 6 | | Control | | SpCas9 | — | | No | N/A |
| 7 | | Test | | SpCas9 | Cas9 gRNA | | Yes. | NA |
| | | | | | | | Partial | |
| 8 | | Control | | SpCas9 | Cas12a gRNA | | No | N/A |
| 9 | ssDNA | Control | M13mp18 | — | — | 10 mM | N/A | No |
| 10 | | Control | | SpCas9 | — | | N/A | No |
| 11 | | Test | | SpCas9 | Cas9 gRNA | | N/A | Yes |
| 12 | | Control | | SpCas9 | Cas12a gRNA | | N/A | No |
| 13 | ssDNA | Control | M13mp18 | — | — | 0.02 mM | N/A | No |
| 14 | | Control | | SpCas9 | — | | N/A | No |
| 15 | | Test | | SpCas9 | Cas9 gRNA | | N/A | No |
| 16 | | Control | | SpCas9 | Cas12a gRNA | | N/A | No |
| 17 | dsDNA + | Control | Zm7.1 + | — | — | 10 mM | No | No |
| | ssDNA | | M13mp18 | | | | | |
| 18 | | Control | | SpCas9 | — | | No | No |
| 19 | | Test | | SpCas9 | Cas9 gRNA | | Yes | Yes |
| 20 | | Control | | SpCas9 | Cas12a gRNA | | No | No |
| 21 | dsDNA + | Control | Zm7.1 + | — | — | 0.2 mM | No | No |
| | ssDNA | | M13mp18 | | | | | |
| 22 | | Control | | SpCas9 | — | | No | No |
| 23 | | Test | | SpCas9 | Cas9 gRNA | | Yes | No |
| 24 | | Control | | SpCas9 | Cas12a gRNA | | No | No |

### Example 9: MgCl₂ Titration Assays

To establish buffer formulation to reduce ssDNase activity of CRISPR nucleases while maintaining the desire dsDNA cutting activity, MgCl₂ titrations were carried out. 1604 nM purified LbCas12a or SpCas9 were assembled with or without the nuclease-appropriate gRNA (100 μM) and incubated with the dsDNA (26.7 nM), M13 ssDNA (12.54 nM), or a combination of dsDNA and ssDNA in cleavage buffer comprising 20 mM HEPES and 0.5 mM DTT. For the titration assays, the cleavage buffer was supplemented with increasing concentrations of MgCl₂ as shown in Tables 17-22. Since the protein concentration was kept constant, the specific protein to DNA ratio was 60:1 for nuclease:dsDNA; 128:1 for nuclease:ssDNA and 40:1 for nuclease:dsDNA+ssDNA. The reactions were carried out at 37° C. for 45 minutes and quenched with proteinase K treatment at 65° C. for 15 minutes. The samples were separated and analyzed on a 1.8% TBE Agarose gel. The observations for LbCas12a are summarized in Tables 17-19 and the observations for SpCas9 are summarized in Tables 20-22.

TABLE 17
MgCl₂ titration assays testing dsDNA cleavage activity of LbCas12a on Zm7.1 dsDNA
template at 60:1 protein to DNA ratio. For targeted dsDNA cleavage, “Yes (Complete)” refers to
the observation of only the ~382 bp and ~1318 bp DNA fragments on the gel. “Yes (Partial)” refers
to the observation of ~1700 bp, ~382 bp and ~1382 bp fragments “No” refers to the observation
of only full length ~1700 bp Zm7.1DNA.

| | | | | MgCl₂ | |
|---|---|---|---|---|---|
| | | | gRNA | (mM) | Targeted |
| | Nuclease | Type of | (Cas12a | conc in | dsDNA |
| Assay/Lane | (LbCas12a) | assay | gRNA) | buffer | cleavage |
| 1 | − | Control | + | 0 | No |
| 2 | + | Control | − | 0.02 | No |
| 3 | + | Test | + | 0.02 | Yes. Partial |
| 4 | + | Test | + | 0.5 | Yes. Complete |
| 5 | + | Test | + | 1 | Yes. Complete |
| 6 | + | Test | + | 2 | Yes. Complete |
| 7 | + | Test | + | 4 | Yes. Complete |
| 8 | + | Test | + | 8 | Yes. Complete |
| 9 | + | Test | + | 10 | Yes. Complete |
| 10 | + | Test | + | 12 | Yes. Complete |
| 11 | + | Test | + | 14 | Yes. Complete |
| 12 | + | Test | + | 16 | Yes. Complete |
| 13 | + | Test | + | 18 | Yes. Complete |
| 14 | + | Test | + | 20 | Yes. Complete |

TABLE 18
MgCl₂ titration assays testing ssDNA cleavage activity of LbCas12a on M13 ssDNA
template at 128:1 Protein to DNA ratio. For ssDNase activity, “Yes. Partial” refers to the
observation that M13mp18 ssDNA band was present but the intensity was less than that observed
in the controls. “Yes. Complete” refers to the observation that M13mp18 ssDNA band was
absent “No” refers to observation that M13mp18 ssDNA band intensity was comparable to the
intensity observed in the controls.

| | | | | MgCl2 | |
|---|---|---|---|---|---|
| | | | gRNA | (mM) | |
| | Nuclease | Type of | (Cas12a | conc in | ssDNA |
| Assay/Lane | (LbCas12a) | assay | gRNA) | buffer | degradation |
| 1 | − | Control | + | 0 | No |
| 2 | + | Control | − | 0.02 | No |
| 3 | + | Test | + | 0.02 | No |
| 4 | + | Test | + | 0.5 | Yes. Partial |
| 5 | + | Test | + | 1 | Yes. Partial |
| 6 | + | Test | + | 2 | Yes. Partial |
| 7 | + | Test | + | 4 | Yes. Complete |
| 8 | + | Test | + | 8 | Yes. Complete |
| 9 | + | Test | + | 10 | Yes. Complete |
| 10 | + | Test | + | 12 | Yes. Complete |
| 11 | + | Test | + | 14 | Yes. Complete |
| 12 | + | Test | + | 16 | Yes. Complete |
| 13 | + | Test | + | 18 | Yes. Complete |
| 14 | + | Test | + | 20 | Yes. Complete |

TABLE 19
MgCl₂ titration assays testing DNA cleavage activity of LbCas12a in samples
comprising Zm7.1 dsDNA and M13 ssDNA template at 40:1 protein: DNA ratio. For targeted
dsDNA cleavage, “Yes (Complete)” refers to the observation of only the ~382 bp and ~1318 bp
DNA fragments on the gel. “Yes (Partial)” refers to the observation of ~1700 bp, ~382 bp and ~1382
bp fragments. “No” refers to the observation of only full length ~1700 bp Zm7.1DNA. For
ssDNase activity, “Yes. Partial” refers to the observation that M13mp18 ssDNA band was present
but the intensity was less than that observed in the controls. “Yes. Complete” refers to the
observation that M13mp18 ssDNA band was absent. “No” refers to observation that M13mp18
ssDNA band intensity was comparable to the intensity observed in the controls.

| | | | | MgCl₂ | | |
|---|---|---|---|---|---|---|
| | | | gRNA | (mM) | Targeted | |
| | Nuclease | Type of | (Cas12a | conc in | dsDNA | ssDNA |
| Assay/Lane | (LbCas12a) | assay | gRNA) | buffer | cleavage | degradation |
| 1 | − | Control | + | 0 | No | No |
| 2 | + | Control | − | 0.02 | No | No |
| 3 | + | Test | + | 0.02 | Yes. Partial | No |
| 4 | + | Test | + | 0.5 | Yes. Complete | Yes. Partial |
| 5 | + | Test | + | 1 | Yes. Complete | Yes. Partial |
| 6 | + | Test | + | 2 | Yes. Complete | Yes. Complete |
| 7 | + | Test | + | 4 | Yes. Complete | Yes. Complete |
| 8 | + | Test | + | 8 | Yes. Complete | Yes. Complete |
| 9 | + | Test | + | 10 | Yes. Complete | Yes. Complete |
| 10 | + | Test | + | 12 | Yes. Complete | Yes. Complete |
| 11 | + | Test | + | 14 | Yes. Complete | Yes. Complete |
| 12 | + | Test | + | 16 | Yes. Complete | Yes. Complete |
| 13 | + | Test | + | 18 | Yes. Complete | Yes. Complete |
| 14 | + | Test | + | 20 | Yes. Complete | Yes. Complete |

TABLE 20
MgCl₂ titration assays testing dsDNA cleavage activity of SpCas9 on Zm7.1 dsDNA
template at 60:1 protein to DNA ratio. For targeted dsDNA cleavage, “Yes. Complete” refers to
the observation of only the ~350 bp and ~1350 bp DNA fragments on the gel. “Yes. Partial” refers
to the observation of ~1700 bp, ~350 bp and ~1350 bp fragments. “No” refers to the observation
of only the full length ~1700 bp Zm7.1DNA

| | | | | MgCl₂ | |
|---|---|---|---|---|---|
| | | | gRNA | (mM) | Targeted |
| | Nuclease | Type of | (Cas12a | conc in | dsDNA |
| Assay/Lane | (SpCas9) | assay | gRNA) | buffer | cleavage |
| 1 | + | Control | − | 0.02 | No |
| 2 | + | Test | + | 0.02 | Yes. Partial |
| 3 | + | Test | + | 0.5 | Yes. Complete |
| 4 | + | Test | + | 1 | Yes. Complete |
| 5 | + | Test | + | 2 | Yes. Complete |
| 6 | + | Test | + | 4 | Yes. Complete |
| 7 | + | Test | + | 8 | Yes. Complete |
| 8 | + | Test | + | 10 | Yes. Complete |
| 9 | + | Test | + | 12 | Yes. Complete |
| 10 | + | Test | + | 14 | Yes. Complete |
| 11 | + | Test | + | 16 | Yes. Complete |
| 12 | + | Test | + | 18 | Yes. Complete |
| 13 | + | Test | + | 20 | Yes. Complete |

TABLE 21
MgCl₂ titration assays testing ssDNA cleavage activity of SpCas9 on M13 ssDNA
template at 128:1 Protein to DNA ratio. For ssDNase activity, “Yes. Partial” refers to the
observation that M13mp18 ssDNA band was present but the intensity was less than that observed
in the controls. “Yes. Complete” refers to the observation that M13mp18 ssDNA band was absent.
“No” refers to observation that M13mp18 ssDNA band intensity was comparable to the intensity
observed in the controls.

| | | | | MgCl₂ | |
|---|---|---|---|---|---|
| | | | gRNA | (mM) | |
| | Nuclease | Type of | (Cas12a | conc in | ssDNA |
| Assay/Lane | (LbCas12a) | assay | gRNA) | buffer | degradation |
| 1 | + | Control | − | 0.02 | No |
| 2 | + | Test | + | 0.02 | No |
| 3 | + | Test | + | 0.5 | Yes. Partial |
| 4 | + | Test | + | 1 | Yes. Partial |
| 5 | + | Test | + | 2 | Yes. Partial |
| 6 | + | Test | + | 4 | Yes. Complete |
| 7 | + | Test | + | 8 | Yes. Complete |
| 8 | + | Test | + | 10 | Yes. Complete |
| 9 | + | Test | + | 12 | Yes. Complete |
| 10 | + | Test | + | 14 | Yes. Complete |
| 11 | + | Test | + | 16 | Yes. Complete |
| 12 | + | Test | + | 18 | Yes. Complete |
| 13 | + | Test | + | 20 | Yes. Complete |

TABLE 22
MgCl₂ titration assays testing DNA cleavage activity of SpCas9 in samples comprising
Zm7.1 dsDNA and Ml3 ssDNA template at 40:1 protein: DNA ratio. For targeted dsDNA
cleavage, “Yes.Complete” refers to the observation of only the ~350 bp and ~1350 bp DNA
fragments on the gel. “Yes.Partial” refers to the observation of ~1700 bp, ~350 bp and ~1350 bp
fragments. “No” refers to the observation of only full length ~1700 bp Zm7.1DNA. For ssDNase
activity, “Yes. Partial” refers to the observation that M13mp18 ssDNA band was present but the
intensity was less than that observed in the controls. “Yes. Complete” refers to the observation that
M13mp18 ssDNA band was absent. “No” refers to observation that M13mp18 ssDNA band
intensity was comparable to the intensity observed in the controls.

| | | | | MgCl₂ | | |
|---|---|---|---|---|---|---|
| | | | gRNA | (mM) | Targeted | |
| | Nuclease | Type of | (Cas12a | conc in | dsDNA | ssDNA |
| Assay/Lane | (LbCas12a) | assay | gRNA) | buffer | cleavage | degradation |
| 1 | + | Control | − | 0.02 | No | No |
| 2 | + | Test | + | 0.02 | Yes. Partial | No |
| 3 | + | Test | + | 0.5 | Yes. Complete | Yes. Partial |
| 4 | + | Test | + | 1 | Yes. Complete | Yes. Partial |
| 5 | + | Test | + | 2 | Yes. Complete | Yes. Complete |
| 6 | + | Test | + | 4 | Yes. Complete | Yes. Complete |
| 7 | + | Test | + | 8 | Yes. Complete | Yes. Complete |
| 8 | + | Test | + | 10 | Yes. Complete | Yes. Complete |
| 9 | + | Test | + | 12 | Yes. Complete | Yes. Complete |
| 10 | + | Test | + | 14 | Yes. Complete | Yes. Complete |
| 11 | + | Test | + | 16 | Yes. Complete | Yes. Complete |
| 12 | + | Test | + | 18 | Yes. Complete | Yes. Complete |
| 13 | + | Test | + | 20 | Yes. Complete | Yes. Complete |

Taken together, the data from Tables 17-22 suggest that lowering the Mg concentration in cleavage buffer can reduce nonspecific ssDNase activity of CRISPR nucleases.

### Example 10: EDTA Chelation Assays

To establish buffer formulations to reduce ssDNase activity of CRISPR nucleases while maintaining the desire dsDNA cutting activity, EDTA titration assays were carried out. Ethylene diamine tetra acetic acid (EDTA) is a chelating agent that can sequester metal ions like Mg2+. Purified LbCas12a or SpCas9 were assembled with or without the nuclease-appropriate gRNA (100 μM) and incubated with the dsDNA (26.7 nM), M13 ssDNA (12.54 nM), or a combination of dsDNA and ssDNA in cleavage buffer comprising 20 mM HEPES, 0.5 mM DTT and 10 mM MgCl₂. For the titration assays, the cleavage buffer was supplemented with increasing concentrations of EDTA as shown in Tables 23-28. The protein amounts were adjusted to accommodate the specific protein to DNA ratio of 60:1 for each reaction. The reactions were carried out at 37° C. for 45 minutes and quenched with proteinase K treatment at 65° C. for 15 minutes. The samples were separated and analyzed on a 1.8% TBE Agarose gel. The observations for LbCas12a are summarized in Tables 23-25 and the observations for SpCas9 are summarized in Tables 26-28.

Taken together, the data from Tables 23-28 suggest that addition of EDTA in cleavage buffer comprising Mg2+ can reduce nonspecific ssDNase activity of CRISPR nucleases.

TABLE 23
EDTA titration assays testing dsDNA cleavage activity of LbCas12a on Zm7.1 dsDNA
template at 10 mM MgCl₂. For targeted dsDNA cleavage, “Yes. Complete” refers to the
observation of only the ~382 bp and ~1318 bp DNA fragments on the gel. “Yes. Partial” refers to
the observation of ~1700 bp, ~382 bp and ~1382 bp fragments. “No” refers to the observation of
only full length ~1700 bp Zm7.1DNA.

| | | | | EDTA | |
|---|---|---|---|---|---|
| | | | gRNA | (mM) | Targeted |
| | Nuclease | Type of | (Cas12a | conc in | dsDNA |
| Assay/Lane | (LbCas12a) | assay | gRNA) | buffer | cleavage |
| 1 | − | Control | − | 0.0 | No |
| 2 | + | Control | − | 0.0 | No |
| 3 | + | Test | + | 0.0 | Yes. Complete |
| 4 | + | Test | + | 0.1 | Yes. Complete |
| 5 | + | Test | + | 1 | Yes. Complete |
| 6 | + | Test | + | 5 | Yes. Complete |
| 7 | + | Test | + | 10 | Yes. Partial |
| 8 | + | Test | + | 15 | Yes. Partial |
| 9 | + | Test | + | 20 | Yes. Partial |

TABLE 24
EDTA titration assays testing ssDNA cleavage activity of LbCas12a on M13 ssDNA at
10 mM MgCl₂. For ssDNase activity, “Yes. Partial” refers to the observation that M13mp18
ssDNA band was present but the intensity was less than that observed in the controls. “Yes.
Complete” refers to the observation that M13mp18 ssDNA band was absent. “No” refers to
observation that M13mp18 ssDNA band intensity was comparable to the intensity observed in
the controls.

| | | | | EDTA | |
|---|---|---|---|---|---|
| | | | gRNA | (mM) | |
| | Nuclease | Type of | (Cas12a | conc in | ssDNA |
| Assay/lane | (LbCas12a) | assay | gRNA) | buffer | degradation |
| 1 | − | Control | − | 0.0 | No |
| 2 | + | Control | − | 0.0 | No |
| 3 | + | Test | + | 0.0 | Yes. Complete |
| 4 | + | Test | + | 0.1 | Yes. Complete |
| 5 | + | Test | + | 1 | Yes. Complete |
| 6 | + | Test | + | 5 | Yes. Complete |
| 7 | + | Test | + | 10 | Yes. Partial |
| 8 | + | Test | + | 15 | Yes. Partial |
| 9 | + | Test | + | 20 | Yes. Partial |

TABLE 25
EDTA titration assays testing DNA cleavage activity of LbCas12a in samples
comprising Zm7.1 dsDNA and M13 ssDNA template at 10 mM MgCl₂. For targeted dsDNA
cleavage, “Yes. Complete” refers to the observation of only the ~382 bp and ~1318 bp DNA
fragments on the gel. “Yes. Partial” refers to the observation of ~1700 bp, ~382 bp and ~1382 bp
fragments. “No” refers to the observation of only full length ~1700 bp Zm7.1DNA. For ssDNase
activity, “Yes. Partial” refers to the observation that M13mp18 ssDNA band was present but the
intensity was less than that observed in the controls. “Yes. Complete” refers to the observation
that M13mp18 ssDNA band was absent. “No” refers to observation that M13mp18 ssDNA band
intensity was comparable to the intensity observed in the controls.

| | | | | EDTA | | |
|---|---|---|---|---|---|---|
| | | | gRNA | (mM) | Targeted | |
| | Nuclease | Type of | (Cas12a | conc in | dsDNA | ssDNA |
| Assay/Lane | (LbCas12a) | assay | gRNA) | buffer | cleavage | degradation |
| 1 | − | Control | − | 0.0 | No | No |
| 2 | + | Control | − | 0.0 | No | No |
| 3 | + | Test | + | 0.0 | Yes. Complete | Yes. Complete |
| 4 | + | Test | + | 0.1 | Yes. Complete | Yes. Complete |
| 5 | + | Test | + | 1 | Yes. Complete | Yes. Complete |
| 6 | + | Test | + | 5 | Yes. Complete | Yes. Complete |
| 7 | + | Test | + | 10 | Yes. Partial | Yes. Partial |
| 8 | + | Test | + | 15 | Yes. Partial | Yes. Partial |
| 9 | + | Test | + | 20 | Yes. Partial | Yes. Partial |

TABLE 26
EDTA titration assays testing dsDNA cleavage activity of SpCas9 on Zm7.1 dsDNA
template at 10 mM MgCl₂. For targeted dsDNA cleavage, “Yes. Complete” refers to the
observation of only the ~350 bp and ~1350 bp DNA fragments on the gel. “Yes. Partial” refers to
the observation of ~1700 bp, ~350 bp and ~1350 bp fragments. “No” refers to the observation of
only full length ~1700 bp Zm7.1DNA.

| | | | | EDTA | |
|---|---|---|---|---|---|
| | | | | (mM) | Targeted |
| | Nuclease | Type of | gRNA | conc in | dsDNA |
| Assay/Lane | (SpCas9) | assay | (Cas9 gRNA) | buffer | cleavage |
| 1 | − | Control | − | 0.0 | No |
| 2 | + | Control | − | 0.0 | No |
| 3 | + | Test | + | 0.0 | Yes. Partial |
| 4 | + | Test | + | 0.1 | Yes. Partial |
| 5 | + | Test | + | 1 | Yes. Partial |
| 6 | + | Test | + | 5 | Yes. Partial |
| 7 | + | Test | + | 10 | No |
| 8 | + | Test | + | 15 | No |
| 9 | + | Test | + | 20 | No |

TABLE 27
EDTA titration assays testing ssDNA cleavage activity of SpCas9a on M13 ssDNA
template at 128:1 Protein to DNA ratio at 10 mM MgCl₂. For ssDNase activity, “Yes. Partial” refers
to the observation that M13mp18 ssDNA band was present but the intensity was less than that
observed in the controls. “Yes. Complete” refers to the observation that M13mp18 ssDNA band
was absent. “No” refers to observation that M13mp18 ssDNA band intensity was comparable to
the intensity observed in the controls.

| | | | | EDTA | |
|---|---|---|---|---|---|
| | | | | (mM) | |
| | Nuclease | Type of | gRNA | conc in | ssDNA |
| Assay/Lane | (SpCas9) | assay | (Cas9 gRNA) | buffer | degradation |
| 1 | − | Control | − | 0.0 | No |
| 2 | + | Control | − | 0.0 | No |
| 3 | + | Test | + | 0.0 | Yes. Partial |
| 4 | + | Test | + | 0.1 | Yes. Partial |
| 5 | + | Test | + | 1 | Yes. Partial |
| 6 | + | Test | + | 5 | Yes. Partial |
| 7 | + | Test | + | 10 | No |
| 8 | + | Test | + | 15 | No |
| 9 | + | Test | + | 20 | No |

TABLE 28
EDTA titration assays testing DNA cleavage activity of SpCas9 in samples comprising
Zm7.1 dsDNA and M13 ssDNA template at 10 mM MgCl₂. For targeted dsDNA cleavage,
“Yes (Complete)” refers to the observation of only the ~350 bp and ~1350 bp DNA fragments on
the gel. “Yes (Partial)” refers to the observation of ~1700 bp, ~350 bp and ~1350 bp fragments.
“No” refers to the observation of only full length ~1700 bp Zm7.1DNA. For ssDNase activity,
“Yes. Partial” refers to the observation that M13mp18 ssDNA band was present but the intensity
was less than that observed in the controls. “Yes. Complete” refers to the observation that
M13mp18 ssDNA band was absent. “No” refers to observation that M13mp18 ssDNA band
intensity was comparable to the intensity observed in the controls.

| | | | | EDTA | |
|---|---|---|---|---|---|
| | | | | (mM) | |
| | Nuclease | Type of | gRNA | conc in | ssDNA |
| Assay/Lane | (SpCas9) | assay | (cas9 gRNA) | buffer | degredation |
| 1 | − | Control | − | 0.0 | No |
| 2 | + | Control | − | 0.0 | No |
| 3 | + | Test | + | 0.0 | Yes. Partial |
| 4 | + | Test | + | 0.1 | Yes. Partial |
| 5 | + | Test | + | 1 | Yes. Partial |
| 6 | + | Test | + | 5 | Yes. Partial |
| 7 | + | Test | + | 10 | No |
| 8 | + | Test | + | 15 | No |
| 9 | + | Test | + | 20 | No |

1. An engineered RNA-guided CRISPR nuclease comprising at least one mutation in a DNA catalytic domain, wherein the engineered RNA guided CRISPR nuclease exhibits reduced non-specific cleavage of single-stranded DNA (ssDNA) as compared to the reference wildtype RNA-guided CRISPR nuclease lacking the at least one mutation.

2. A method of creating an engineered RNA-guided CRISPR nuclease comprising editing a polynucleotide encoding a wildtype RNA-guided CRISPR nuclease to generate at least one mutation in a DNA catalytic domain, wherein the engineered RNA-guided CRISPR nuclease exhibits reduced non-specific cleavage of single-stranded DNA as compared to the wildtype RNA-guided CRISPR nuclease lacking the at least one mutation.

3. A method of reducing non-specific single-stranded DNA (ssDNA) cleavage caused by an RNA-guided CRISPR nuclease, comprising providing a cell with an engineered RNA-guided CRISPR nuclease comprising at least one mutation in a DNA catalytic domain as compared to a reference wildtype RNA-guided CRISPR nuclease, wherein the engineered RNA guided CRISPR nuclease exhibits reduced non-specific cleavage of a non-target ssDNA as compared to the reference wildtype RNA-guided CRISPR nuclease lacking the at least one mutation.

4. The engineered RNA-guided CRISPR nuclease of claim 1, wherein the engineered RNA-guided CRISPR nuclease is part of a ribonucleoprotein.

5. The engineered RNA-guided CRISPR nuclease of claim 4, wherein the ribonucleoprotein comprises at least one guide nucleic acid.

6. The engineered RNA-guided CRISPR nuclease of claim 1, wherein the engineered RNA-guided CRISPR nuclease is a Cas12a nuclease.

7. The engineered RNA-guided CRISPR nuclease of claim 1, wherein the engineered RNA-guided CRISPR nuclease is a Cas12a nuclease, and the wildtype RNA-guided CRISPR nuclease comprises the amino acid sequence of SEQ ID NO: 2.

8. The engineered RNA-guided CRISPR nuclease of claim 1, wherein the engineered RNA-guided CRISPR nuclease is selected from the group consisting of a Cas9 nuclease, a CasX nuclease, a CasY nuclease, and a C2c2 nuclease.

9. The engineered RNA-guided CRISPR nuclease of claim 1, wherein the engineered RNA-guided CRISPR nuclease comprises an amino acid sequence selected from the group consisting of SEQ ID NOs: 7-12.

10. The engineered RNA-guided CRISPR nuclease of claim 1, wherein the engineered RNA-guided CRISPR nuclease exhibits the ability to cleave double-stranded DNA (dsDNA).

11. The engineered RNA-guided CRISPR nuclease of claim 10, wherein the engineered RNA-guided CRISPR nuclease cleaves dsDNA at a rate that is at least 50% of the cleavage rate of the cleavage rate of the wildtype RNA-guided CRISPR nuclease.

12. The engineered RNA-guided CRISPR nuclease of claim 1, wherein the DNA catalytic domain comprises a domain selected from the group consisting of a RuvC domain, a Nuc domain, and an HNH domain.

13. The engineered RNA-guided CRISPR nuclease of claim 1, wherein the at least one mutation is selected from the group consisting of an insertion, a deletion, and a substitution.

14. The engineered RNA-guided CRISPR nuclease of claim 6, wherein the Cas12a nuclease comprises a substitution of an amino acid at a position selected from the group consisting of position 925 and position 1138 as compared to SEQ ID NO: 2.

15. The engineered RNA-guided CRISPR nuclease of claim 1, wherein the reduced cleavage of ssDNA exhibits a reduced rate of cleavage as compared to the wildtype RNA-guided CRISPR nuclease.

16. The engineered RNA-guided CRISPR nuclease of claim 1, wherein the reduced cleavage of ssDNA comprises a ssDNA cleavage rate that is less than 50% of the ssDNA cleavage rate of the wildtype RNA-guided CRISPR nuclease.

17. The engineered RNA-guided CRISPR nuclease of claim 1, wherein the reduced cleavage of ssDNA is measured within 180 minutes of introducing the engineered RNA-guided CRISPR nuclease to ssDNA.

18. The engineered RNA-guided CRISPR nuclease of claim 1, wherein the engineered RNA-guided CRISPR nuclease cleaves dsDNA in a eukaryotic cell.

19. The engineered RNA-guided CRISPR nuclease of claim 18, wherein the eukaryotic cell is selected from the group consisting of a plant cell, an animal cell, a protozoan cell, and a fungal cell.

20.-31. (canceled)


SEQUENCE LISTING (23 sequences)

SEQ ID NO: 1 - DNA, 1707 bp, Zea mays
tggatgtacagagtgatattattgacacgccatgcagataccaagcggcctctagaggatccaggagcaaccgtgagcaagggcgaggagaataacatggccatcatcaggagttcatgcgcttcaaggtgcgcatggagggctccgtgaacggccacgaggaggggcccctacgagggcttagaccgctagctgaaggtgaccaagggtggcatattacccatatcttcaagttggacatcacctcccacaacgaggactacaccatcgtggaacagtacgaacgcgccgagggcgagctcgcggcaagggcgaggaactgttcactgccggccagcatttgaaacatggcctttagtataatatgatggcatgccctcttgcaatgtatgaaccaaggtggggtaggagctgtactcgatagcccgggcttcaagtgggagcgcgtgatgctaggaactgtactccatcgcctagcgcatttccaagggagcctaaaggtagaagcgcatcgagggagagaggtgaagctgcgactcctccgagcggatgtcccctatcgtacggaggaacgggccggcagggaccaacttcatgtaggagatcaagatgaggctgaagctgaaggacggcggccaccctccgaggtcagaccacctacactcggccaggagcccgtgcagcgtggcacctcgattgacatgtggggagctatattcgatagcctagtaaaaacgaatcgagggagaacgatgataacgtggtcccaatcctggtggaactggatggtgatgtgaacgggcacaagttctccgtcagcggagagggtgaaccctgaagttcatctgcactaccgggaagctccctgttccgtggcctaccctcgtcaccaccttcacctacggtgttcagtgcttctcccggtatccagatcacatgaagcagcatgacttcttcaagagcgccatgcccgaaggctacgtgcaagagaggactatctcgttcaaggatgacgggaactacaagacacgtgccgaagtcaagttcgaaggtgataccctggtgaaccgcatcgagctgaagggtatcgacttcaaggaagatggcaacatcctcggacacaagctggagtacaactacaactcccacaacgtatacatcacggccgacaagcagaagaacggcatcaaggctaacttcaagatcaggggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacacaacatcgaagatggaagcgtgcaactggcggaccactaccagcagaacacgcccatcggcgatggccctgtcctgctgccggacaaccattacctgtccacgcaatctgccctctccaaggaccccaacgagaagagggaccacatggtcctgctggagttcgtgacggctgctgggatcacgcatggcatggatgaactctacaagtaggcggtctgataaaacagaatttgcctggcg

SEQ ID NO: 2 - protein, 1229 aa, Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHA

SEQ ID NO: 3 - DNA, 3687 bp, Artificial Sequence
  Synthetic sequence
atgagcaaactggaaaaattcaccaactgttactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgttgcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatgtttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgattgccacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctatgatgtatataaagataaacgtttctccgaagatcagtacgaactgcacatcccgatcgcaattaacaaatgcccgaaaaacatcttcaaaatcaacaccgaagtgcgtgttctgctgaaacacgatgataacccgtacgttattggcatcgaccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatctgtgaactggttgaaaaatacgatgcagttatcgcgctggaagatctgaacagcggtttcaaaaactcacgtgtaaaagttgaaaaacaggtttaccagaaattcgaaaaaatgctgattgataaactgaactatatggtggataaaaaatctaacccgtgcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgtgcctgaccagcgcgtataaagaactgttcaacaaatacggcattaactaccagcagggcgacattcgtgcgctgctgtgtgaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaagcatgca

SEQ ID NO: 4 - DNA, 33 bp, Artificial Sequence
  Synthetic sequence
atgggcagcagccatcatcaccaccatcaccat

SEQ ID NO: 5 - DNA, 30 bp, Solanum tuberosum
ggtagcaaaaagaggcgtatcaagcaggac

SEQ ID NO: 6 - DNA, 30 bp, Lycopersicon esculentum
ggatctaagaagcgtaggatcaagcaagat

SEQ ID NO: 7 - protein, 1229 aa, Artificial Sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHA

SEQ ID NO: 8 - protein, 1229 aa, Artificial Sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMANSITGRTDVDFLISPVKNSDGIFYDSRNYEAQENAILPKNADANGAYNIARKVLWAIGQFKKAEDEKLDKVKIAISNKEWLEYAQTSVKHA

SEQ ID NO: 9 - protein, 1229 aa, Artificial Sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHA

SEQ ID NO: 10 - protein, 1229 aa, Artificial Sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMHNSITGRTDVDFLISPVKNSDGIFYDSRNYEAQENAILPKNADANGAYNIARKVLWAIGQFKKAEDEKLDKVKIAISNKEWLEYAQTSVKHA

SEQ ID NO: 11 - protein, 1229 aa, Artificial Sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMQNSITGRTDVDFLISPVKNSDGIFYDSRNYEAQENAILPKNADANGAYNIARKVLWAIGQFKKAEDEKLDKVKIAISNKEWLEYAQTSVKHA

SEQ ID NO: 12 - protein, 1229 aa, Artificial Sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMENSITGRTDVDFLISPVKNSDGIFYDSRNYEAQENAILPKNADANGAYNIARKVLWAIGQFKKAEDEKLDKVKIAISNKEWLEYAQTSVKHA

SEQ ID NO: 13 - protein, 1229 aa, Artificial Sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTAVDFLISPVKNSDGIFYDSRNYEAQENAILPKNADANGAYNIARKVLWAIGQFKKAEDEKLDKVKIAISNKEWLEYAQTSVKHA

SEQ ID NO: 14 - protein, 1229 aa, Artificial Sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTSVDFLISPVKNSDGIFYDSRNYEAQENAILPKNADANGAYNIARKVLWAIGQFKKAEDEKLDKVKIAISNKEWLEYAQTSVKHA

SEQ ID NO: 15 - protein, 1229 aa, Artificial Sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTCVDFLISPVKNSDGIFYDSRNYEAQENAILPKNADANGAYNIARKVLWAIGQFKKAEDEKLDKVKIAISNKEWLEYAQTSVKHA

SEQ ID NO: 16 - protein, 1229 aa, Artificial Sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTEVDFLISPVKNSDGIFYDSRNYEAQENAILPKNADANGAYNIARKVLWAIGQFKKAEDEKLDKVKIAISNKEWLEYAQTSVKHA

SEQ ID NO: 17 - protein, 1229 aa, Artificial Sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVAFLISPVKNSDGIFYDSRNYEAQENAILPKNADANGAYNIARKVLWAIGQFKKAEDEKLDKVKIAISNKEWLEYAQTSVKHA

SEQ ID NO: 18 - protein, 1229 aa, Artificial Sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVSFLISPVKNSDGIFYDSRNYEAQENAILPKNADANGAYNIARKVLWAIGQFKKAEDEKLDKVKIAISNKEWLEYAQTSVKHA

SEQ ID NO: 19 - protein, 1229 aa, Artificial Sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVCFLISPVKNSDGIFYDSRNYEAQENAILPKNADANGAYNIARKVLWAIGQFKKAEDEKLDKVKIAISNKEWLEYAQTSVKHA

SEQ ID NO: 20 - protein, 1229 aa, Artificial Sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVEFLISPVKNSDGIFYDSRNYEAQENAILPKNADANGAYNIARKVLWAIGQFKKAEDEKLDKVKIAISNKEWLEYAQTSVKHA

SEQ ID NO: 21 - RNA, 44 nt, Artificial Sequence
  Synthetic sequence
guaauuucuacucuuguagauguauaauaugauggcaugcccuc

SEQ ID NO: 22 - protein, 1368 aa, Streptococcus pyogenes [sequence omitted]

SEQ ID NO: 23 - DNA, 4104 bp, Artificial Sequence
  Synthetic sequence
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgac